(19) United States
(12) Patent Application Publication
(10) Pub. No.: US 2026/0226077 A1
(43) Pub. Date: Aug. 6, 2026

(54) TYPE II TOPOISOMERASE INHIBITORS AND METHODS OF MAKING AND USING THEREOF

(71) Applicant: Ohio State Innovation Foundation, Columbus, OH (US)

(72) Inventor: Mark MITTON-FRY, Columbus, OH (US)

(21) Appl. No.: 19/156,807

(22) PCT Filed: Feb. 16, 2024
(86) PCT No.: PCT/US2024/016130

§ 371 Date: Aug. 14, 2025

Related U.S. Application Data
(60) Provisional application No. 63/446,110, filed on Feb. 16, 2023.

(51) Int. Cl. C07D 519/00; A61K 31/5383; A61K 31/542; A61P 31/04

(52) U.S. Cl. CPC C07D 519/00; A61K 31/5383; A61K 31/542; A61P 31/04

(57) ABSTRACT

Disclosed are Type II Topoisomerase Inhibitors, analogs thereof, pharmaceutical compositions thereof, and methods of making and using these compounds and compositions. Methods of using the disclosed compounds to treat infections, such as MRSA are also described.

### CROSS REFERENCE TO RELATED APPLICATIONS

This application claims the benefit of priority to U.S. Provisional Application 63/446,110, filed Feb. 16, 2023, which is incorporated by reference herein in its entirety.

### BACKGROUND

It has been estimated that there were 4.95 million deaths associated with bacterial antimicrobial resistance in 2019, among them 1.27 million directly attributable deaths (Murray, C. J. L., et al. Lancet 2022, 399, 629). Thus, infections caused by multidrug-resistant pathogens such as methicillin-resistant Staphylococcus aureus (MRSA) and Pseudomonas aeruginosa present a critical threat to human health. Surveillance of antibacterial susceptibility among clinical isolates continues to reveal new mechanisms of resistance, including to antibiotics traditionally considered the last lines of defense, such as carbapenems (Morrill, H. J., et al. Open For. Infect. Dis. 2015, 2, ofv050) and even colistin (Liu, Y-Y., et al. Lancet Infect. Dis. 2016, 16, 161; Schwarz, S., et al. J. Antimicrob. Chemother. 2016, 71, 2066).

Despite tremendous advances in genetics and high-throughput screening, the development of antibiotics against novel biological targets has proven exceptionally difficult. The advancement of next-generation inhibitors from established classes of antibiotics such as fluoroquinolones likewise presents difficulties, e.g., preexisting resistance and clinical efficacy/safety differentiation. In light of these and other challenges, antibacterial research in the pharmaceutical industry, long a source of new medicines, has dwindled substantially. Advances in other areas of medicine, including cancer therapy, surgery, and organ transplantation, critically require the availability of effective antibiotics. The confluence of diminished private investment and rising resistance to life-saving medications thus presents an extraordinary threat, to individual patients and to modern medicine. In order to avoid a post-antibiotic era (Alanis, A. J. Arch. Med. Res. 2005, 36, 697), new antibacterial therapies and new approaches to preventing, diagnosing, and treating infections are desperately needed.

Bacteria are historically grouped into two categories based on their cellular structure, and this distinction helps to guide the diagnosis and treatment of bacterial infections. Gram-positive (G-pos.) bacteria possess a comparatively thick peptidoglycan cell wall, and Gram-negative (G-neg.) bacteria possess a thinner peptidoglycan layer further surrounded by an outer membrane. Both categories give rise to life-threatening infections, and both have been associated with the emergence of multidrug resistance. Enterococcus faecium and Methicillin Resistant Staphylococcus aureus (MRSA) represent important examples of G-pos. pathogens. The Centers for Disease Control and Prevention (CDC) estimates that MRSA alone causes >10,000 fatalities per year in the United States. G-neg. pathogens include Klebsiella pneumoniae, Acinetobacter baumannii, Pseudomonas aeruginosa, and various Enterobacter species among many others. Together, these six types of G-pos. and G-neg. bacteria, known by the acronym ESKAPE pathogens, comprise some of the most significant causes of multidrug-resistant (MDR) infections today. Effective antibacterial therapy typically abrogates a process that bacteria require to survive (an “essential” target). Such processes include the synthesis and maintenance of proteins, DNA, and the bacterial cell wall (Kohanski, M. A., et al. Nature Rev. Microbiol. 2010, 8, 423). These targets are largely intracellular, meaning that most antibiotics need to penetrate the bacterial cell in order to exert their effect. The outer membrane of G-neg. bacteria constitutes a particularly significant barrier to cell entry, and scientists currently lack a sufficient understanding of the molecular features that govern G-neg. cell penetration (vide infra) (Scorciapino, M. A., et al. Future Med. Chem. 2016, 8, 1047). This fact, coupled with the robust set of resistance mechanisms employed by G-neg. microorganisms (Ruppe, E., et al. Ann. Intensive Care 2015, 5, 21), renders the cure of infections caused by these bacteria particularly challenging.

DNA replication is essential for the survival of bacteria, and the heterotetrameric (A2B2) bacterial type II topoisomerases DNA gvrase and Topoisomerase IV (TopoIV) are important to this process (Mayer, C., et al. Chem. Rev. 2014, 114, 2313; Collin, F., et al. Appl. Microbiol. Biotechnol. 2011, 92, 479; Bisacchi, G. S., et al. Annual Rep. Med. Chem. 2009, 44, 379; Black, M. T., et al. Curr. Opin. Invest. Drugs 2009, 10, 804; Bradbury, B. J., et al. Curr. Opin. Pharmacol. 2008, 8, 574; Mitscher, L. A. Chem. Rev. 2005, 105, 559: Hooper. D. C. Clin. Inf Dis. 2000, 31 (Suppl. 2), S24). The widely used fluoroquinolone (FQ) class of antibiotics targets this mechanism. The commercially successful and medically important FQ class of antibacterial agents exemplifies the enormous potential of disrupting these targets and reveals the possibility of achieving both Gram-positive and Gram-negative spectrum (Bax, B. D., et al. Nature 2010, 466, 935). However. FQ resistance has emerged via a number of mechanisms (Blumberg, H. M., et al. J. Inf Dis. 1991, 163, 1279; Hooper, D. C. Emerging Inf Dis. 2001, 7, 337; Jacoby, G. A. Clin. Infect. Dis. 2005, 41 (Suppl. 2), S120). The history of the FQs has also informed more recent drug discovery strategies, notably a focus on minimization of hERG inhibition (a cardiotoxicity liability) and the design of dual-targeting (gyrase and TopoIV) inhibitors to slow clinical resistance emergence (Id.; Blumberg, H. M., et al. J. Inf Dis. 1991, 163, 1279; Wohlkonig, A.; Chan, P. F., et al. Nat. Struct. Mol. Biol. 2010, 17, 1152; Aldred, K. J., et al. Biochem. 2014, 53, 1565; Onodera. Y., et al. J. Antimicrob. Chemother. 1999, 44, 533; Cheng, J., et al. Antimicrob. Agents Chemother. 2007, 51, 2445; Morrow, B. J., et al. Antimicrob. Agents Chemother. 2011, 55, 5512). Given exhaustive efforts directed toward FQs over several decades, as well as multiple mechanisms of resistance in the clinic (Hooper, D. C. Emerging Inf Dis. 2001, 7, 337: Jacoby, G. A. Clin. Infect. Dis. 2005, 41 (Suppl. 2). S120), the design and synthesis of type II topoisomerase inhibitors with non-FQ chemotypes presents a compelling need. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to Non-FQ Bacterial Type II Topoisomerase Inhibitors (NBTIs), analogs thereof, pharmaceutical compositions thereof, and methods of making and using these compounds and compositions. In further aspects, the disclosed subject matter relates to NBTIs with both gyrase and TopoIV activity, analogs thereof, pharmaceutical compositions thereof, and methods of making and using these compounds and compositions. The disclosed compounds can have potent and balanced inhibition of gyrase and TopoIV (to maximize bacterial killing and slow resistance emergence), minimal hERG inhibition (to reduce cardiotoxicity liabilities), and physicochemical properties consistent with desirable pharmacokinetic (PK) properties.

Methods of using the disclosed compounds to treat infections, such as MRSA, MDR P. aeruginosa, and other pathogens are also described herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a graph of in vivo efficacy data for compound 284 in a mouse septicemia model.

FIG. 2 is a graph of in vivo efficacy data for compound 284 against S. aureus infection.

### DETAILED DESCRIPTION

The materials, compounds, compositions, and methods described herein may be understood more readily by reference to the following detailed description of specific aspects of the disclosed subject matter, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, reference to “the kinase” includes mixtures of two or more such kinases, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction.” is meant lowering of an event or characteristic (e.g., bacterial growth or infection). It is understood that this is typically in relation to some standard or expected value, in other words it is relative, but that it is not always necessary for the standard or relative value to be referred to. For example, “reduces bacterial growth” means decreasing the amount of bacteria cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of alleviation of one or more symptoms (such as bacterial growth or infection), diminishment of extent of infection, stabilized (i.e., not worsening) state of infection, preventing or delaying spread of the infection, preventing or delaying occurrence or recurrence of infection, delay or slowing of infection progression, and amelioration of the infected state.

The term “patient” preferably refers to a human in need of treatment for any purpose, and more preferably a human in need of a treatment to treat infection. However, the term “patient” can also refer to non-human animals, preferably mammals such as dogs, cats, horses, cows, pigs, sheep and non-human primates, among others, that are in need of treatment with a compound as disclosed herein.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol, as described below.

The symbols Aⁿ is used herein as merely a generic substituent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond.

Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can have from 5 to 14 carbon atoms. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H.

Throughout this specification “C(O)” is a short hand notation for C═O, which is also referred to as oxo.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O—.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above. The term “sulfoxide” is used herein to refer to the sulfo-oxo group represented by the formula —OS(O)₂A′, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

As used herein, the symbol

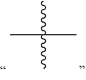

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

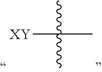

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

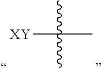

infers that when R³ is “XY”, the point of attachment bond is the same bond as the bond by which R³ is depicted as being bonded to CH₃.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind.

Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to infection, an effective amount comprises an amount sufficient to cause a bacterial cell to shrink and/or to decrease the growth rate of the bacterial cells or to prevent or delay other unwanted infection. In some embodiments, an effective amount is an amount sufficient to delay development. In some embodiments, an effective amount is an amount sufficient to prevent or delay occurrence and/or recurrence.

An effective amount can be administered in one or more doses. In the case of bacterial infection, the effective amount of the drug or composition may: (i) reduce the number of bacterial cells: (ii) reduce bacterial cell size; (iii) inhibit, retard, slow to some extent and preferably stop bacterial cell infiltration into peripheral organs; (iv) inhibit bacterial growth; (vi) prevent or delay occurrence and/or recurrence of bacterial infection; and/or (vii) relieve to some extent one or more of the symptoms associated with the infection.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples.

### Compounds

Examples of some NBTIs in the literature are provided in Scheme 1. As illustrated by GSK299423 (1) (Bax, B. D., et al. Nature 2010, 466, 935) and summarized by Singh (Singh, S. B., et al. ACS Med. Chem. Lett. 2014, 5, 609), NBTIs share three common structural domains: a) a left-hand side (LHS) usually comprising a fused bicyclic or tricyclic ring system, b) a linker domain with an amine positioned to interact with D83 of gyrase, and c) a right-hand side (RHS) comprising an aromatic or heteroaromatic ring. X-ray crystallography has been used to study the binding of these compounds to a complex of gyrase and DNA. This research has provided insight at the molecular level into compound binding (Widdowson, K., et al. Future Med. Chem. 2010, 2, 1619; Lahiri, S. D., et al. Antimicrob. Agents Chemother. 2015, 59, 5278), and this understanding has been enhanced through the study of target mutations conferring resistance to NBTIs.

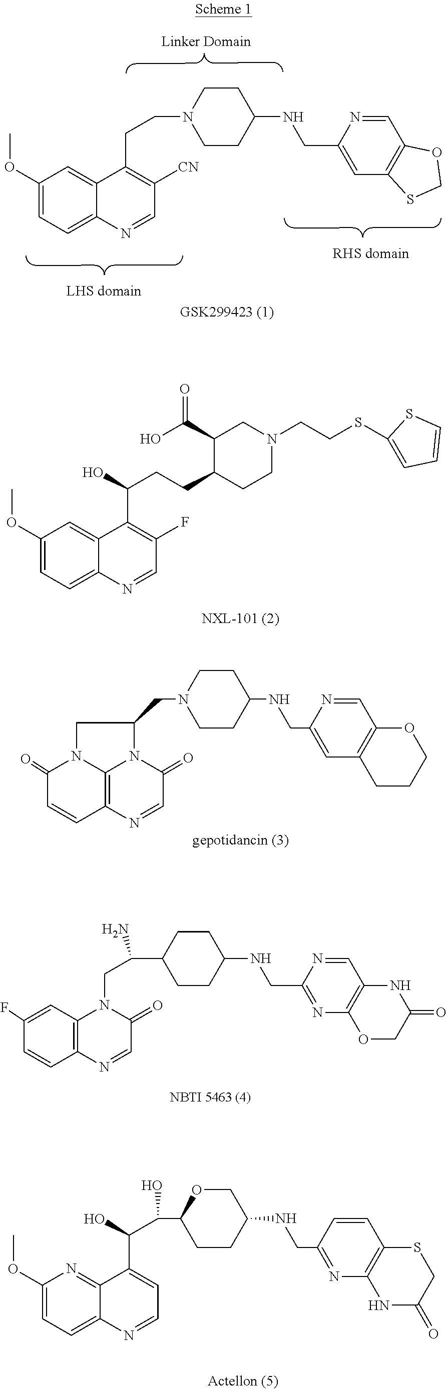 [SMILES: COc1ccc2ncc(C#N)c(CCN3CCC(NCc4cc5c(cn4)OCS5)CC3)c2c1 ; COc1ccc2ncc(F)c([C@@H](O)CC[C@@H]3CCN(CCSc4cccs4)C[C@@H]3C(=O)O)c2c1 ; O=c1ccc2ncc(=O)n3c2n1C[C@H]3CN1CCC(NCc2cc3c(cn2)OCCC3)CC1 ; N[C@@H](Cn1c(=O)cnc2ccc(F)cc21)C1CCC(NCc2ncc3c(n2)OCC(=O)N3)CC1 ; COc1ccc2nccc([C@@H](O)[C@H](O)[C@@H]3CC[C@@H](NCc4ccc5c(n4)NC(=O)CS5)CO3)c2n1]

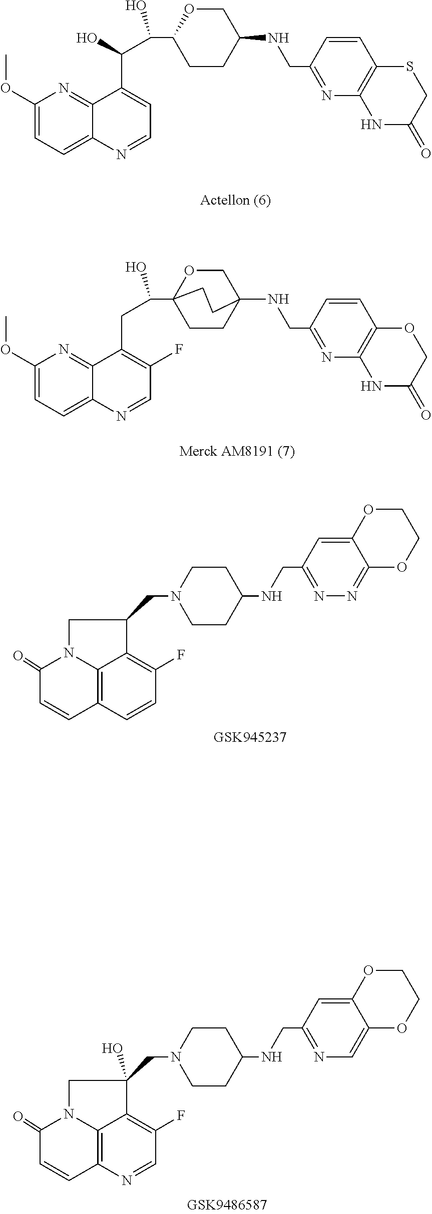 [SMILES: COc1ccc2nccc([C@@H](O)[C@H](O)[C@H]3CC[C@H](NCc4ccc5c(n4)NC(=O)CS5)CO3)c2n1 ; COc1ccc2ncc(F)c(C[C@H](O)C34CCC(NCc5ccc6c(n5)NC(=O)CO6)(CC3)CO4)c2n1 ; O=c1ccc2ccc(F)c3c2n1C[C@H]3CN1CCC(NCc2cc3c(nn2)OCCO3)CC1 ; O=c1ccc2ncc(F)c3c2n1C[C@@]3(O)CN1CCC(NCc2cc3c(cn2)OCCO3)CC1]

The LHS binds with uncleaved DNA, and the RHS, generally containing an aromatic or heteroaromatic ring, binds to a dimeric interface of gyrase. Commonly observed gyrase mutations conferring resistance to NBTLs, such as substitutions at D83 and M121, occur at this interface. Extensive previous efforts have optimized the LHS and RHS moieties and illustrated the tolerance for structural variety and innovation in the linker (Mitton-Frv, M. J. Med. Chem. Rev. 2017, 52, 281; Tan, C. M., et al. Antimicrob. Agents Chemother. 2016, 60, 4830; Black. M. T., et al. Antimicrob. Agents Chemother. 2008, 52, 3339; Mitton-Fry. M. J., et al. Bioorg. Med. Chem. Lett. 2013, 23, 2955; Dougherty, T. J., et al. Antimicrob. Agents Chemother. 2014, 58, 2657; Dougherty, T. J., et al. Antimicrob. Agents Chemother. 2014, 58, 4250; Nayar, A. S., et al. Antimicrob. Agents Chemother. 2015, 59, 331; Reck, F., et al. Bioorg. Med. Chem. 2014, 22, 5392; Surivet, J-P., et al. J. Med. Chem. 2013, 56, 7396; Surivet. J-P., et al. J. Med. Chem. 2015, 58, 927: Miles, T. J., et al. Bioorg. Med. Chem. Lett. 2011, 21, 7489: Wiles, J. A., et al. J. Med. Chem. 2011, 54, 3418; Mitton-Fry, M. J. Novel, Nonquinolone Inhibitors of DNA Gyrase and Topoisomerase IV: Antibacterial Activity and Resistance Mechanisms. Presented at the 243rd National Meeting of the American Chemical Society, San Diego, CA, 2012, Paper MEDI-257; Singh, S. B., et al. Bioorg. Med. Chem. Lett. 2015, 25, 2409; Singh, S. B., et al. Bioorg. Med. Chem. Lett. 2015, 25, 3636; Singh, S. B., et al. Med. Chem. Commun. 2015, 6, 1773; So, W., et al. Antimicrob. Agents Chemother. 2015, 59, 4956; Miles, T. J., et al. Bioorg. Med. Chem. Lett. 2013, 23, 5437).

The largely solvent-exposed linker domain serves to bridge the LHS and RHS and does not itself play a critical role in binding, evidenced by the linker diversity tolerated in compounds 1-7. In the compounds disclosed herein, a new linker moiety is introduced, which has been found to modulate the physicochemical properties. The structural simplicity and synthetic accessibility of the linker moiety disclosed herein can also result in improved synthetic efficiency and cost effectiveness. Specifically, the disclosed compounds have a 5-amino-1,3-dioxane linker moiety, shown below.

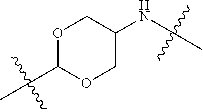 [SMILES: *NC1COC(*)OC1]

The 1,3-dioxane represents a rather uncommon design feature, owing to concerns about potential hydrolytic instability of the acetal. However, in the disclosed compounds, this linker moiety can reduce lipophilicity and amine basicity, improving pharmacokinetic and cardiac safety properties (Pasternak, A., et al. Bioorg. Med. Chem. Lett. 1999, 9, 491; Ndubaku, C. O., et al. ACS Med. Chem. Lett. 2015, 6, 1241; Li, L., et al. Bioorg. Med. Chem. Lett. 2018, 28, 2477).

In some aspects, disclosed herein are compounds that are Type II Topoisomerase Inhibitors having Formula I.

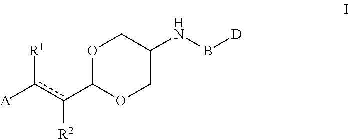

wherein
the dashed line represents a bond that is present or absent, and when the bond is present, R¹ and R² can be cis or trans:
A is a fused bicyclic aryl or bicyclic heteroaryl ring optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, phosphate, or thiol; or A and R¹ together form a tricyclic ring optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, phosphate, or thiol;
B is C₁-C₆ alkyl or C₄-C₆ cycloalkyl optionally substituted with one or more oxo, alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, phosphate, or thiol;
D is a bicyclic heteroaryl ring having Formula IV as detailed herein:
R¹ and R² are, independently, chosen from H, OH, Cl, F, Br, I, CN, NO₂, NH₂, CF₃, CO₂H, CO₂NH₂, CO₂NHR³, CO₂R³, C(O)R³, C(O)NH₂, C(O)NHR³, oxo (i.e., ═O), and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, phosphate, or thiol; or R¹ is a C₁-C₃ alkyl or C₂-C₃ alkenyl, optionally substituted with R¹⁰, also bound to A;
each R³ is, independently, chosen from C₁-C₆ alkyl, C₁-C₆ cycloalkyl, aryl, heteroaryl, heterocycloalkyl, and heteroalkyl, any of which are optionally substituted with C₁-C₆ alkyl, C₁-C₆ alkoxyl, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol; and
R¹⁰ is H, Cl, F, Br, I, CN, OH, NO₂, NH₂, CF₃, CO₂H, CO₂NH₂, CO₂NHR³, CO₂R³. C(O)R³, C(O)NH₂, C(O)NHR³, or C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
or a pharmaceutically acceptable salt thereof.

In specific examples of Formula I, the dashed line represents a bond and Formula I can thus be represented as Formula IA (R¹ and R² are trans) or IB (R¹ and R² are cis).

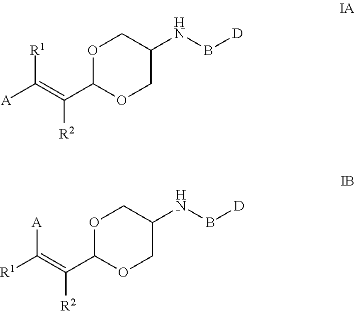

In still other examples, the dashed line in Formula I is absent and Formula I can thus be represented as Formula IC.

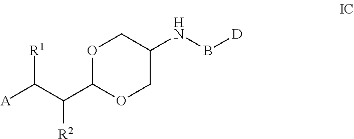

In additional examples, the stereochemistry of the dioxane moiety can be trans, which is shown in Formula ID.

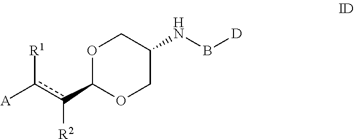

In still other examples, the stereochemistry of the dioxane moiety can be cis. In each of these formulas, the variables R¹, R², R³, A, B, D are as defined herein. Further, unless specifically stated, reference to Formula I herein includes individual references to each of Formulas IA-ID.

In specific examples herein, R¹ is OH and R² is H in Formulas I, IA, IB, IC, or ID. In other examples, R¹ is H and R² is OH in Formulas I, IA, IB, IC, or ID.

The disclosed compounds can have potent and balanced inhibition of gyrase and TopoIV (to maximize bacterial killing and slow resistance emergence), minimal hERG inhibition (to reduce cardiotoxicity liabilities), and physicochemical properties consistent with desirable pharmacokinetic (PK) properties (Lipinski, C. A., et al. Adv. Drug Delivery Rev. 1997, 23, 3; Veber, D. F., et al. J Med. Chem. 2002, 45, 2615; Gleeson, M. P. J Med. Chem. 2008, 51, 817: Leeson, P. D., et al. Nature Rev. Drug Disc. 2007, 6, 881), and ease of synthesis. Further, these compounds can utilize a mechanistically distinct form of topoisomerase inhibition resulting in antibacterial activity even against highly FQ-resistant strains.

Previous work with NBTIs has helped to clarify their advantages and remaining challenges. The interactions with the target, distinct from those of FQs, lead to a lack of cross-resistance between these two classes of topoisomerase inhibitors (Black, M. T., et al. Antimicrob. Agents Chemother. 2008, 52, 3339; Mitton-Fry, M. J.; Brickner, S. J., et al. Bioorg. Med. Chem. Lett. 2013, 23, 2955). As such, NBTIs do not face the issue of widespread preexisting resistance in the clinic that would be encountered with a novel FQ. Excellent efficacy against Gram-positive pathogens such as MRSA, both in vitro (Minimum Inhibitory Concentrations, MICs) and in vivo (murine models of infection) has been demonstrated for structurally diverse NBTIs. More recent work has also suggested that an appropriately situated primary amine in the linker domain, such as that found in NBTI 5463 (4, Scheme 1) may be sufficient for antibacterial activity against critically important Gram-negative pathogens such as P. aeruginosa (Dougherty, T. J., et al. Antimicrob. Agents Chemother. 2014, 58, 2657; Dougherty, T. J., et al. Antimicrob. Agents Chemother. 2014, 58, 4250; c) Nayar, A. S., et al. Antimicrob. Agents Chemother. 2015, 59, 331) potentially as a result of improved porn penetration. Such effects have also been observed with amine incorporation in other antibacterial drug classes such as cephalosporins.

Among the challenges associated with NBTIs, two deserve special attention, hERG inhibition, with attendant concern about QT-prolongation and cardiovascular safety, must be closely monitored (Reck, F., et al. Bioorg. Med. Chem. 2014, 22, 5392: Miles, T. J., et al. Bioorg. Med. Chem. Lett. 2011, 21, 7483: Geng, B., et al. Bioorg. Med. Chem. Lett. 2011, 21, 5432; Reck, F., et al. J. Med Chem. 2011, 54, 7834; Reck, F., et al. J. Med Chem. 2012, 55, 6916; Wiles, J. A., et al. J. Med. Chem. 2011, 54, 3418; Singh, S. B., et al. Bioorg. Med. Chem. Lett. 2015, 25, 1831; Singh, S. B., et al. Bioorg. Med. Chem. Lett. 2015, 25, 2473). At least one clinical candidate, NXL-101 (2, Scheme 1) (Black, M. T., et al. Antimicrob. Agents Chemother. 2008, 52, 3339) was withdrawn from clinical studies as a result of QT-prolongation. Historically, NBTIs demonstrate superior inhibition of gyrase as compared to TopoIV, at least in S. aureus, permitting resistance by means of single-step mutations to the gyrase target. Improved inhibition of TopoIV has been associated with diminished resistance (Surivet, J-P., et al. J. Med. Chem. 2013, 56, 7396; Surivet, J-P., et al. J. Med Chem. 2015, 58, 927).

It has been demonstrated that hERG inhibition from NBTIs often correlates strongly with lipophilicity and amine basicity. That is, while lipophilicity increases so does hERG inhibition. While not wishing to be bound by theory, compounds with a 5-amino-1,3-dioxane linker moiety were prepared to minimize hERG inhibition via reduced amine basicity and lipophilicity (Ndubaku, C. O., et al. ACS Med. Chem. Lett. 2015, 6, 1241; Li. L., et al. Bioorg. Med Chem. Lett. 2018, 28, 2477; Lu, Y., et al. J. Med. Chem. 2021, 64, 15214; Lu, Y., et al. ACS Med. Chem. Lett. 2022, 13, 955) and provide ready synthetic accessibility across a wide range of derivatives (U.S. Pat. No. 11,352,349). It was found, however, that some of those compounds with 5-amino-1,3-dioxane linker moieties, and thus with reduced lipophilicity as compared to other NBTIs, hERG inhibition actually increased (see FIG. 6 of Lu Y. J. Med. Chem. 64:15215, 2021). It has been surprisingly found herein that a new group of NBTIs with 5-amino-1,3-dioxane linker moieties that is even more hydrophilic that those previously prepared defies this trend and has reduced hERG inhibition.

Aside from reducing amine basicity and lipophilicity, the readily accessible achiral dioxane linker also enhances synthetic efficiency compared to tetrahydropyran (THP) and oxabicyclooctane linkers (Scheme 1). THPs 5, 6, and oxabicylooctane 7 all display excellent antibacterial activity, reinforcing the tolerance for structural changes to the linker, provided that the overall molecular topology is maintained. However, synthesis of the linker alone for 7 required 14 steps, and 5 and 6 suffer from synthetic and stereochemical complexity.

In addition to structural diversity, the disclosed compounds can be used to explore a breadth of physicochemical properties, including CLogP and topological polar surface area (TPSA). Variations in LHS, linker substitution, and RHS can be explored systematically. The LHS plays a key role in interacting with DNA. Quinoline LHS A (Scheme 1) has been used successfully by several teams (Wiles, J. A., et al. J. Med. Chem. 2011, 54, 3418; Mitton-Fry, M. J. Novel, Non-quinolone Inhibitors of DNA Gyrase and Topoisomerase IV: Antibacterial Activity and Resistance Mechanisms. Presented at the 243rd National Meeting of the American Chemical Society, San Diego, CA, 2012, Paper MEDI-257), and 1,5-naphthyridine B (Scheme 1) has likewise seen extensive usage (Singh, S. B., et al. Bioorg. Med. Chem. Lett. 2015, 25, 2409: Singh. S. B., et al. Bioorg. Med. Chem. Lett. 2015, 25, 3636; Singh, S. B., et al. Med. Chem. Commun. 2015, 6, 1773). LHS C (Scheme 1) dramatically reduces the lipophilicity of the planned analogs (ca. 2 CLogP units versus A) and has been shown to provide potent analogs in several reports. Substitution of the methoxv group of LHS C with fluorine, as in LHS D (Scheme 1), has been shown previously to reduce the undesired inhibition of cardiac ion channels, and D is the core for the promising Gram-negative lead NBTI 5463. Additionally, tricyclic LHS moieties such as LHS E-H (Scheme 1) have also shown promise (Miles, T. J., et al. Bioorg. Med Chem. Lett. 2013, 23, 5437; Singh, S. B., et al. Bioorg. Med. Chem. Lett. 2015, 25, 1831: Singh, S. B., et al. Bioorg. Med Chem. Lett. 2015, 25, 2473; Miles, T. J., et al. Bioorg. Med Chem. Lett. 2016, 26, 2464; Biedenbach, D. J., et al. Antimicrob. Agents Chemother. 2016, 60, 1918).

In view of the above, specific examples disclosed herein are compounds of Formula I, specifically Formulas IA-ID, wherein A is a fused bicyclic aryl or bicyclic heteroaryl ring

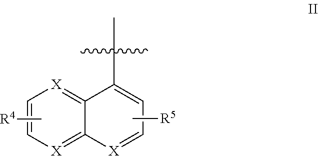

wherein
each X is, independently. CH or N; and
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

In specific examples, A can have Formula II, wherein R¹ and R⁵ are, independently, chosen from H, Cl, F, Br, I, CN, OH, and unsubstituted C₁-C₆ alkyl or C₁-C₆ alkoxyl. In further examples, R⁴ and R⁵ are, independently, chosen from H, Cl, F, CN, OH, and methoxyl. In further examples, R⁴ and R⁵ are, independently, chosen from F and methoxyl. In still further examples, all X's are CH. In yet further examples, one X is CH and the other two X's are N. In yet further examples, two X's are CH and the other X is N. In still further examples, all X's are N.

In a specific example, A is chosen from

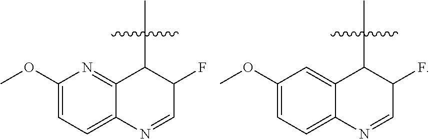

In further examples, disclosed herein are compounds of Formula I, specifically Formulas IA-ID, wherein A is a fused bicyclic aryl or bicyclic heteroaryl ring having Formula III.

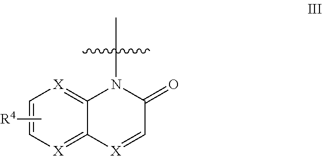

wherein
each X is, independently, CH or N;
R⁴ is chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

In specific examples, A can have Formula III, wherein R⁴ is chosen from H, Cl, F, Br, I, CN, OH, and unsubstituted C₁-C₆ alkyl or C₁-C₆ alkoxyl. In further examples, R⁴ is chosen from H, Cl, F, CN, OH, and methoxyl. In further examples, R⁴ is chosen from F and methoxyl. In specific examples of Formula III, each X is N. In other examples, two X's are CH and the other X is N. In other examples, two X's are N and the other X is CH.

In still further examples, disclosed herein are compounds of Formula I, specifically Formulas IA-ID, wherein A is a bicyclic aryl or bicyclic heteroaryl that together with R¹ forms a tricyclic ring. When R¹ is a CH₂, this can be shown by Formula IX, X, XI, or XII.

In specific examples, A can be Formula IX:

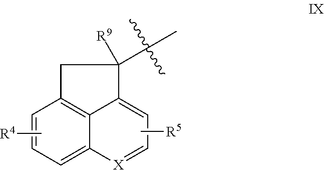

wherein
X is CH, N, or CR⁸;
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
R⁸ is Cl, F, CN, OH, OCH₃, CH₃, or NH₂; and
R⁹ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

In specific examples, A can be Formula X:

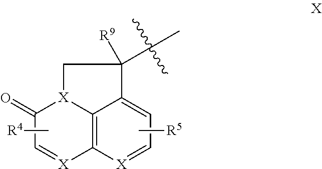

wherein
each X is, independently, CH, N, or CR⁸:
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
each R⁸ is, independently, Cl, F, CN, OH, OCH₃, CH₃, or NH₂; and
R⁹ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

In specific examples, A can be chosen from

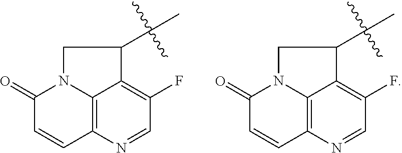

In specific examples, A can be Formula XI

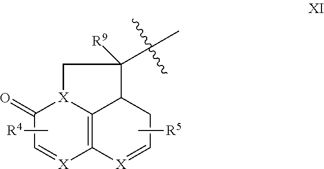

wherein
each X is, independently, CH, N, or CR⁸;
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
each R⁸ is, independently, Cl, F, CN, OH, OCH₃, CH₃, or NH₂; and
R⁹ is H or C₁-C₆ alkyl.

In specific examples, A can be Formula XII:

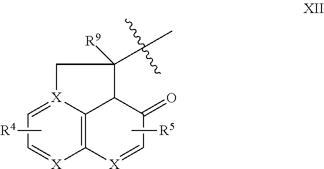

wherein
each X is, independently, CH, N, or CR⁸;
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R, C(O)NH₂, C(O)NHR, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol; and each R⁸ is Cl, F, CN, OH, OCH₃, CH₃, or NH₂; and
R⁹ is H or C₁-C₆ alkyl.

In specific examples, of Formula I, A can be

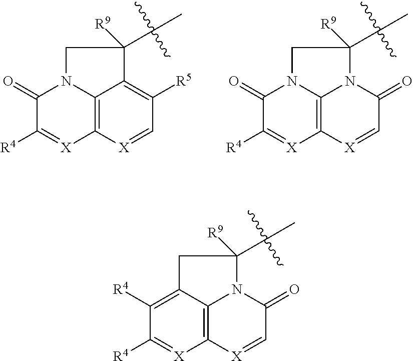

wherein each of R⁴, R⁵, and R⁹ can be independently, chosen from Cl, F, CN, OH, OCH₃, CH₃, or NH₂.
In specific examples, of Formula IX, X, XI, and XII, R⁵ can be F.

In further examples, disclosed herein are compounds of Formula I, specifically Formulas IA-ID, wherein B is a C₁-C₆ alkyl or C₄-C₆ cycloalkyl chosen from unsubstituted methyl, ethyl, propyl, butyl, cyclobutyl, or cyclopentyl. In specific examples, B is CH₂, —C(═O)—, or cyclobutyl. B can also be CONH or CH₂NH—.

In still further examples, disclosed herein are compounds of Formula I, specifically Formulas IA-ID, wherein D is a heteroaryl ring having Formula IV.

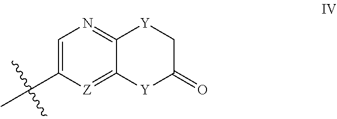

wherein
each Y is, independently, chosen from O, S, NH, or CH₂;
Z is N or CH; and
R⁶ is chosen from H, Cl, F, Br, I, CN, OH, NO₂, NH₂, CF₃, CO₂H. CO₂NH₂, CO₂NHR₃, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

In specific examples, D can have Formula IV, wherein R⁶ is chosen from H, Cl, F, Br, I, CN, OH, and unsubstituted C₁-C₆ alkyl or C₁-C₆ alkoxyl. In further examples, R⁶ is chosen from H, Cl, F, CN, OH, or methoxyl. In further examples, R⁶ is chosen from F or methoxyl. In further examples, R⁶ is H. In still further examples, both Y are O. In other examples, one Y is S and the other is O. In still other examples, one Y is NH and the other is O.

In a specific example, disclosed are compounds of Formula I, where D is Formula IV, with Z being N. In other examples, disclosed are compounds of Formula I, where D is Formula IV and Z is CH.

In yet further examples, disclosed herein are compounds of Formula I, specifically Formulas IA-ID, wherein R¹ and R² are, independently, chosen from H, F, OH, or NH₂. In specific examples, R² is NH₂. In other examples, R² is H or OH. In further examples, R¹ is H or OH. In specific examples, R² is oxo (i.e., ═O). In other examples, R¹ is oxo (i.e., ═O). Incorporation of a hydroxyl substituent (at R¹ or R²) reduces lipophilicity by ca. 1.5 CLogP units and has been shown in some cases to impact hERG inhibition and other properties.

Some specific examples of compounds disclosed herein are shown in the examples and in Scheme 2.

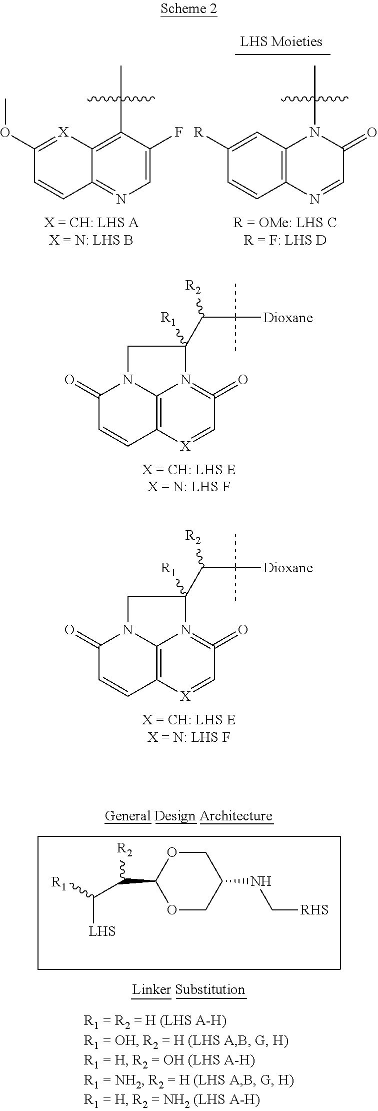

where RHS is formula IV.

Additional examples of compounds disclosed herein are shown below.

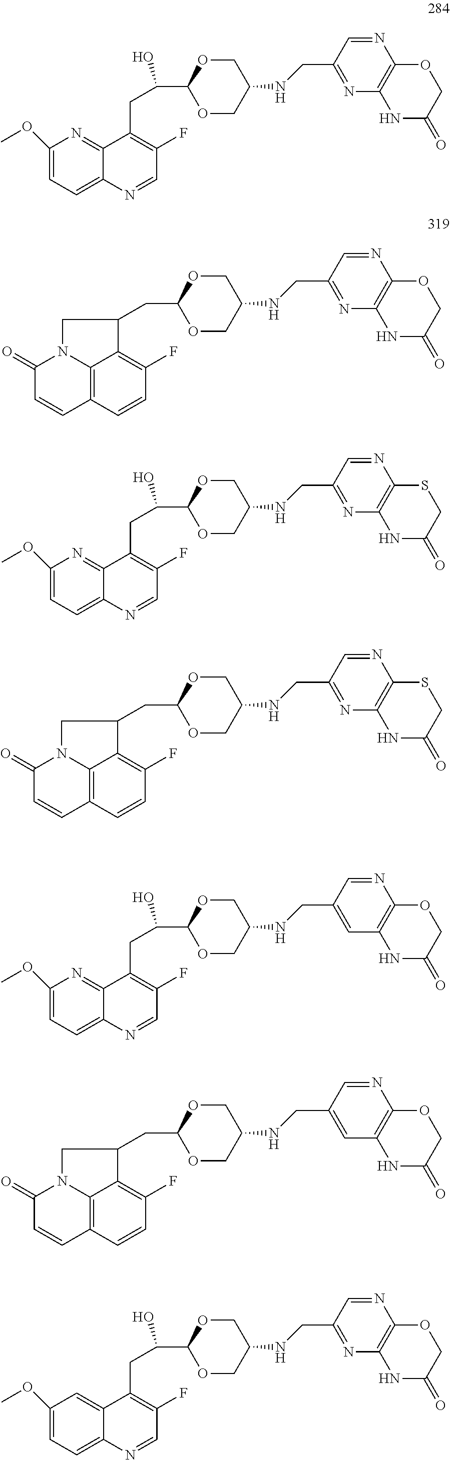

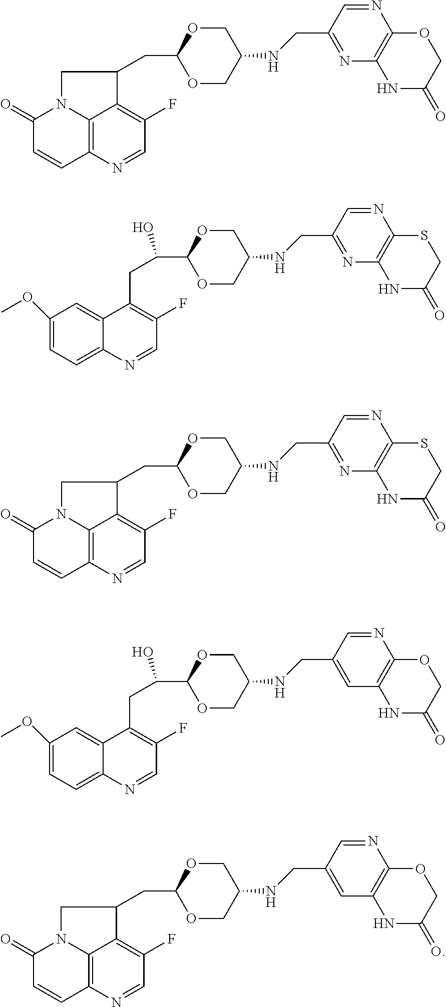 [SMILES: O=C1COc2ncc(CN[C@H]3CO[C@H](CC4Cn5c(=O)ccc6ncc(F)c4c65)OC3)nc2N1 ; COc1ccc2ncc(F)c(C[C@H](O)[C@H]3OC[C@H](NCc4cnc5c(n4)NC(=O)CS5)CO3)c2c1 ; O=C1CSc2ncc(CN[C@H]3CO[C@H](CC4Cn5c(=O)ccc6ncc(F)c4c65)OC3)nc2N1 ; COc1ccc2ncc(F)c(C[C@H](O)[C@H]3OC[C@H](NCc4cnc5c(c4)NC(=O)CO5)CO3)c2c1 ; O=C1COc2ncc(CN[C@H]3CO[C@H](CC4Cn5c(=O)ccc6ncc(F)c4c65)OC3)cc2N1]

### Method of Use

The compounds disclosed herein can be used to treat infections and inhibit the growth of bacteria. In certain examples, disclosed are methods of treating an infection in a patient, comprising administering to the patient a thereapeutically effective amount of any of the compounds disclosed herein. Specific examples of infections that can be treated include, but are not limited to, Actinobacter, Actinomycetes, Bacilli, Bortedellen, Clostridia, Corynebacteria, Enterobacter, Enterococci, Helicobacter, Haemophilus, Klebsiella, Listeria, Mycobacteria, Neisseria, Shigella, Salmonella, tuberculosis bacteria, and Yersinia.

In some examples, the disclosed compounds can be used to treat infections caused by resistant G-pos. bacteria such as Methicillin Resistant Staphylococcus aureus (MRSA). Despite newly launched drugs and others in clinical development, the CDC characterizes MRSA as a serious threat, its second highest level of concern. These disclosed methods can involve administering a compound disclosed herein to the infected human or animal or the human or animal at risk of being infected. In some specific examples, the infected individual has cyctic fibrosis.

In some examples, the disclosed compounds can be used to treat infections caused by resistant G-neg. pathogens such as P. aeruginosa. Infections caused by G-neg. bacteria in general, and MDR P. aeruginosa in particular (Wagner, S., et al. J. Med. Chem. 2016, 59, 5929), represent a key need in antibacterial drug discovery that is currently underrepresented by approaches in clinical development. The additional permeability barrier imposed by the outer membrane of G-neg. organisms (Zgurskaya, H. I., et al. ACS Infect. Dis. 2015, 1, 512), as well as other resistance mechanisms such as robust multidrug efflux transporters, make the identification of potential new therapies particularly challenging. These disclosed methods can involve administering a compound disclosed herein to the infected human or animal or the human or animal at risk of being infected.

In still other examples, the disclosed compounds can be used to treat infections by M. tuberculosis, M. avium, or M abscessus.

In some examples, the disclosed compounds can be used to treat infections caused by Enterococcus faecium, Klebsiella pneumoniae, Acinetobacter baumannii, various Enterobacter, and Neisseria gonorrhoeae. Further examples include the following diseases include: tuberculosis; Pneumonia; Typhoid; Paratyphoid; Syphilis, Gastritis; Gastroenteritis; Ruhr; Pestilence; Enteritis; extraintestinal infections, peritonitis and appendicitis with E. coli and intestinal infections with EHEC, EPEC, ETEC and EIEC; Cholera, Legionnaires' disease, whooping cough, brucellosis, Lyme disease, leptospirosis, typhus, trachoma, gonorrhea, meningitis, septicemia, leprosy etc. These methods can involve administering a compound disclosed herein to the infected human or animal or the human or animal at risk of being infected.

In other examples, disclosed herein are methods of treating an infection in a patient, comprising administering to the patient a therapeutically effective amount of any of the compounds disclosed herein.

In these disclosed methods, one can treat humans with infections, but also can treat livestock (horses, cows, pigs, sheep, goats etc.), poultry, and companion animals (dogs, cats, rabbits, etc.). The compositions or organisms can be administered alone or in combination with other therapeutics or nutritional supplements, for example the composition can be combined into a feed.

### Combinations

The disclosed compounds can also be combined with additional antimicrobial agents. For example, the disclosed compounds can be combined with one or more of Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicvlic acid; Aminosalicvlate sodium; Amoxicillin; Amphomycin; Ampicillin; Ampicillin Sodium; Apalcillin Sodium; Apramycin; Aspartocin; Astromicin Sulfate; Avilamycin: Avoparcin; Azithromycin: Azlocillin; Azlocillin Sodium: Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride: Bispyrithione Magsulfex; Butikacin; Butirosin Sulfate; Capreomvcin Sulfate: Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin: Cefoxitin Sodium: Cefpimizole; Cefpimizole Sodium: Cefpiramide: Cefpiramide Sodium; Cefpirome Sulfate; Cefpodoxime Proxetil: Cefprozil; Cefroxadine; Cefsulodin Sodium; Ceftazidime; Ceftibuten; Ceftizoxime Sodium: Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium: Cephapirin Sodium; Cephradine; Cetocycline Hydrochloride: Cetophenicol; Chloramphenicol: Chloramphenicol Palmitate; Chloramphenicol Pantothenate Complex: Chloramphenicol Sodium Succinate; Chlorhexidine Phosphanilate: Chloroxvlenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium: Cyclacillin; Cycloserine: Dalfopristin: Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline: Doxycycline Calcium; Doxycycline Fosfatex; Doxycycline Hyclate: Droxacin Sodium; Enoxacin; Epicillin; Epitetracycline Hydrochloride; Erythromycin; Erythromycin Acistrate: Erythromycin Estolate; Erythromycin Ethylsuccinate: Erythromycin Gluceptate; Erythromycin Lactobionate; Erythromycin Propionate; Erythromycin Stearate; Ethambutol Hydrochloride: Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin: Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamy cin; Levofuraltadone; Levopropylcillin Potassium; Lexithromycin; Lincomycin; Lincomycin Hydrochloride; Lomefloxacin; Lomefloxacin Hydrochloride: Lomefloxacin Mesylate: Loracarbef; Mafenide; Meclocycline: Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium: Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodiumr; Nafcillin Sodium; Nalidixate Sodium; Nalidixic Acid; Natainycin; Nebramycin; Neomycin Palmitate; Neomycin Sulfate; Neomycin Undecylenate; Netilmicin Sulfate: Neutramycin; Nifuiradene: Nifuraldezone: Nifuratel; Nifuratrone; Nifurdazil; Nifurimide; Nifiupirinol; Nifurquinazol; Nifurthiazole: Nitrocycline: Nitrofurantoin; Nitromide; Norfloxacin; Novobiocin Sodium; Ofloxacin; Onnetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine: Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin: Racephenicol; Ramoplanin; Ranimycin; Relomycin; Repromicin; Rifabutin: Rifametane; Rifamexil; Rifamide; Rifampin; Rifapentine; Rifaximin: Rolitetracycline: Rolitetracycline Nitrate; Rosaramicin: Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole: Sulfamethoxazole: Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisboxazole Diolamine; Sulfomyxin; Sulopenem; Sultamricillin; Suncillin Sodium; Talampicillin Hydrochloride; Teicoplanin; Temafloxacin Hydrochloride: Temocillin; Tetracycline; Tetracycline Hydrochloride; Tetracycline Phosphate Complex; Tetroxoprim; Thiamphenicol: Thiphencillin Potassium; Ticarcillin Cresyl Sodium; Ticarcillin Disodium; Ticarcillin Monosodium: Ticlatone; Tiodonium Chloride; Tobramycin; Tobramycin Sulfate; Tosufloxacin: Trimethoprim; Trimethoprim Sulfate; Trisulfapyrimidines; Troleandomycin; Trospectomycin Sulfate; Tvrothricin: Vancomvcin; Vancomycin Hydrochloride; Virginiamycin; or Zorbamycin.

The disclosed compounds can also be combined with foaming agents such as sodium laureth ether sulfate (SLES), sodium lauryl dodecyl sulfate (SDS), disodium laureth sulfosuccinate, ammonium lauryl sulfate (ALS), sodium pareth sulfate, and sodium coceth sulfate. Foaming agents can be present at from about 1% to about 70%, about 5% to about 50%, about 10% to about 30%, or about 1% to about 5% by weight.

The disclosed compounds can, in some examples, further comprise one or more antibiotics. Examples of antibiotics include amikacin, gentamicin, kanamycin, neomycin, streptomycin, tobramycin, bacitracin, clindamycin, daptomycin, lincomycin, linezolid, metronidazole, polymyxin, rifaximin, vancomycin, penicillin, cephalosporin, cephazolin, cephalexin, erythromycin, azithromycin, ciprofloxacin, levofloxacin, sulfadiazine, minocycline, tetracycline, and rifampin. The proportion of antibiotics can be about 0.001% to about 10%, about 0.01% to about 5%, about 0.1% to about 10%, or about 1% to about 5% by weight.

The disclosed compounds can, in some examples, further comprise additional agents such as acyclovir, cephradine, malphalen, procaine, ephedrine, adriamycin, dauno, mycin, plumbagin, atropine, quinine, digoxin, and quinidine, cephradine, cephalothin, cishydroxy-L-proline, melphalan, nicotinic acid, nitric oxide, nitroglycerin, chemodeoxycholic acid, chlorambucil, paclitaxel, sirolimus, 5-flurouracil, paclitaxel, mercaptoethanesulfonate, verapamil, or antifungal agents. The proportion of these additional agents can be about 0.001% to about 10%, about 0.01% to about 5%, about 0.1% to about 10%, or about 1% to about 5% by weight.

In some examples, the disclosed compounds can further comprise anti-inflammatory agents. Examples of such agents include acetaminophen, aspirin, celecoxib, diclofenac, diflunisal, flurbiprofen, ibuprofen, indomethacin, ketoprofen, ketorolac, meclofenamate, meloxicam, methyl salicylate, nabumetone, naproxen, oxaprozin, piroxicam, sulindac, tolmetin, trolamine. The proportion of these anti-inflammatory agents can be present in the formulation at from about 1% to about 70%, about 5% to about 50%, about 10% to about 30%, or about 1% to about 5% by weight.

### Administration

The disclosed compounds can be administered either sequentially or simultaneously in separate or combined pharmaceutical formulations. When one or more of the disclosed compounds is used in combination with a second therapeutic agent the dose of each compound can be either the same as or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, phosphate, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S.

Application Publication No. 20020035243 also describes compositions for transporting biological moieties across cell membranes for intracellular delivery. Compounds can also be incorporated into polymers, examples of which include poly (D-L lactide-co-glycolide) polymer, poly[bis(p-carboxyphenoxy) propane:sebacic acid] in a 20:80 molar ratio (as used in GLIADEL); chondroitin; chitin; and chitosan.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### General Synthesis

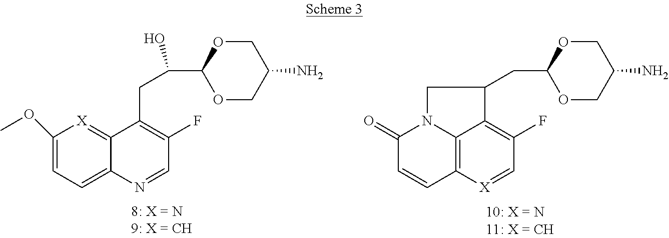

The synthesis of primary amines 8 and 9 has been described in Lu. Y., et al. J. Med. Chem. 2021, 64, 15214. The synthesis of primary amines 10 and 11 has been described in Lu, Y., et al. ACS Med Chem. Lett. 2022, 13, 955.

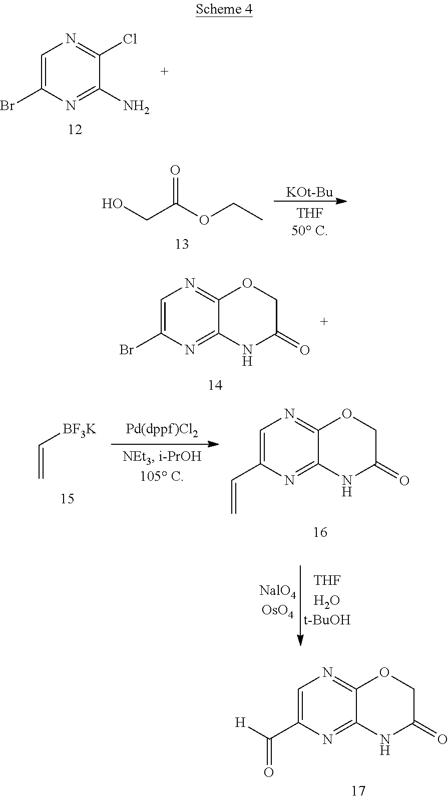

### 6-bromo-2H,3H,4H-pyrazino[2,3-b][1,4]oxazin-3-one (14)

6-bromo-3-chloropyrazin-2-amine (12, 1.5 g. 7.2 mmol, 1 equiv) was taken up in anhydrous THF (72 mL). Ethyl glycolate (13) was added to the reaction mixture by syringe (2.04 mL, 21.6 mmol, 3 equiv) followed by the slow addition of 1M KOt-Bu in THF (18 mL) The solution stirred at 50° C. overnight under nitrogen. The reaction mixture was cooled to room temperature and diluted with H₂O and EtOAc. The phases were separated and the EtOAc layer was extracted with water (100 mL×2) until the aq solution remained clear and slightly yellow in color. The pH of the aqueous layer was adjusted to 4 using 1M HCl. The acidic aqueous solution was extracted with EtOAC (100 mL×5), dried with Na₂SO₄, decanted, and concentrated to afford a brownish orange solid (1.73 g). The solid was taken up in anhydrous THF (50 mL). To it was added 1M KOt-Bu in THF (9 mL). The reaction mixture was stirred at 50° C. for an additional 24 hours, at which point it was cooled and the extraction process and pH adjustment was repeated to afford the title compound as a brownish orange solid (1.4 g, 6.1 mmol, 85%). Alternatively, a second portion of 1M KOt-Bu may be added without the initial aqueous workup. ¹H NMR (DMSO-d₆) δ: 11.84 (br s, 1H); 7.92 (s, 1H); 4.90 (s, 2H).

Alternatively, compound 14 may be prepared as described below:

An oven-dried 3-necked 2 L flask equipped with one waterless reflux condenser was charged with 6-bromo-3-chloropyrazin-2-amine (12, 25.5 g. 122 mmol, 1 equiv) and vac/N₂ purged. Anhydrous THF (564 mL) was added, the resulting pale yellow solution was stirred under N₂, and methyl glycolate (28.0 mL, 32.7 g, 2.97 equiv) was added by syringe. To the solution was added 1M potassium tert-butoxide in THF (300 mL, 300 mmol, 2.5 equiv) dropwise by cannula over approximately one hour. The internal temperature rose from 22.7 to 33.6° C. during the addition, and the color changed first to a brighter yellow, then amber, and then finally to a dark chocolate color. After the addition was complete, the reaction was heated at an internal temperature of 45° C. overnight. The reaction turned somewhat lighter in color during this time, and a fair amount of solid precipitated. Magnetic stirring became difficult as the reaction progressed. After 21.5h, the color was similar but more gray-brown, and there was a considerable amount of precipitated solid in the reaction. Stirring had ceased. An additional portion of 1M potassium tert-butoxide in THF (150 mL, 150 mmol, 1.2 equiv) was added over several minutes while the reaction was still being heated. It was difficult to get effective stirring during this time, necessitating repeated hand swirling of the flask to mix it well. The reaction was still heterogeneous thereafter, and it took on a brown color. The solid was brown in color as well whereas before it was gray. The internal temperature rose during this time to approximately 60° C.; stirring restarted after the addition was complete but remained problematic. The reaction was continued at 50° C. for 9h, whereupon the solvent was removed to near dryness by rotary evaporation to afford a solid residue. The solid was dissolved in 500 mL distilled H₂O to afford a dark brown solution. The solution was treated with 1M HCl until pH ~5; copious amounts of a solid precipitated. The solid was collected by vacuum filtration using a large Buchner funnel and washed portionwise with water totaling 350 mL, affording a sandy-gray colored solid. Drying first on the funnel for 1 h and then overnight in a vacuum desiccator afforded the title compound as a sandy-colored solid (24.16 g, 86%).

### 6-ethenyl-2H,3H,4H-pyrazino[2,3-b][1,4]oxazin-3-one (16)

6-bromo-2H,3H,4H-pyrazino[2,3-b][1,4]oxazin-3-one (14, 1.1 g, 4.8 mmol, 1 equiv), potassium vinyl trifluoroborate (15, 833 mg, 6.22 mmol, 1.3 equiv), and [1,1′-bis(diphenylphosphino)ferrocene]dichloropalladium (II) (78.1 mg, 0.096 mmol, 0.02 equiv) were added to a pressure vial followed by iPOH (44 mL) and triethylamine (0.8 mL, 5.74 mmol, 1.2 equiv). The tightly capped mixture was stirred at 105° C. for 48 hours. The pressure vial was cooled to room temperature and the mixture filtered through a celite plug and concentrated. The crude product was loaded onto silica gel and purified using a 25 g silica gel column (hexanes/EtOAc. 0 to 100% gradient over 30 minutes). The title compound was a shimmery off white solid (0.65 g. 3.7 mmol, 77%). ¹H NMR (DMSO-d₆) δ: 11.59 (br s, 1H); 7.83 (s, 1H); 6, 74 (dd, J=17.3, 10.8 Hz, 1H); 6.07 (dd, J=17.3, 1.8 Hz, 1H); 5.41 (dd, J=10.8, 1.8 Hz, 1H); 4.88 (s, 2H).

### 3-oxo-2H,3H,4H-pyrazino[2,3-b][1,4]oxazine-6-carbaldehyde (17)

6-ethenyl-2H,3H,4H-pyrazino[2,3-b][1,4]oxazin-3-one (16, 22 mg, 0.12 mmol, 1 equiv) was taken up in THF (1.2 mL) and H₂O (1.2 mL). To the stirring solution was added sodium periodate (80 mg, 0.37 mmol, 3 equiv) followed by a small portion of osmium tetroxide in t-butanol (9.6% wt.). The reaction mixture was stirred 1.5 hours and became cloudy white. The mixture was quenched slowly in a dropwise manner with saturated aqueous Na₂S₂O₃. The solution was diluted with EtOAc and H₂O. The phases were separated, and the aqueous layer was extracted with EtOAC (25 mL×5). The combined organic layers were washed with brine, dried with Na₂SO₄, decanted, and concentrated to afford a white solid. The material was passed through a small silica gel plug to remove excess sodium periodate and Na₂SO₄ using EtOAC. The title compound was an off white solid (17 mg, 0.095 mmol, 76%). Alternatively, the crude product can be purified by flash chromatography on silica gel (hexanes/EtOAc, 0 to 50% gradient). ¹H NMR (DMSO-d₆) δ: 11.95 (br s, 1H); 9.87 (s, 1H); 8.34 (s, 1H): 4.98 (s, 2H).

Alternatively, compound 17 can be prepared as described below:

6-ethenyl-2H,3H,4H-pyrazino[2,3-b][1,4]oxazin-3-one (16, 1.90 g, 10.7 mmol, 1 equiv) was dissolved in THF (50 mL) to give light yellow solution. Distilled water was then added and the reaction cooled to 0° C. in ice bath, whereupon the solid largely precipitated. Warmed to room temperature in water bath, still heterogeneous. Potassium osmate dihydrate (82 mg, 0.22 mmol, 0.021 equiv) added, followed immediately thereafter by NaIO₄ (6.9 g, 32 mmol, 3.0 equiv). Within a few minutes, reaction was extremely heterogeneous, and the temperature rose to a maximum of ~25° C. After 60 min. the reaction was quenched by addition of 50 mL sat. aq. Na₂S₂O₃, with initial slow addition. The internal temperature rose to 28° C. Reaction stirred for approximately 15 minutes and then transferred to a separatory funnel. 50 mL H₂O and 50 mL EtOAc added; much solid remained undissolved. Phases separated and aqueous extracted 3×50 mL EtOAc. Combined organics washed 1×25 mL brine, dried over Na₂SO₄, decanted, and concentrated to afford 873.5 mg. Combined aqueous layers were extracted again 3×50 mL EtOAc. Concentration with the previous crude material afforded the title compound (949.3 mg. 49%).

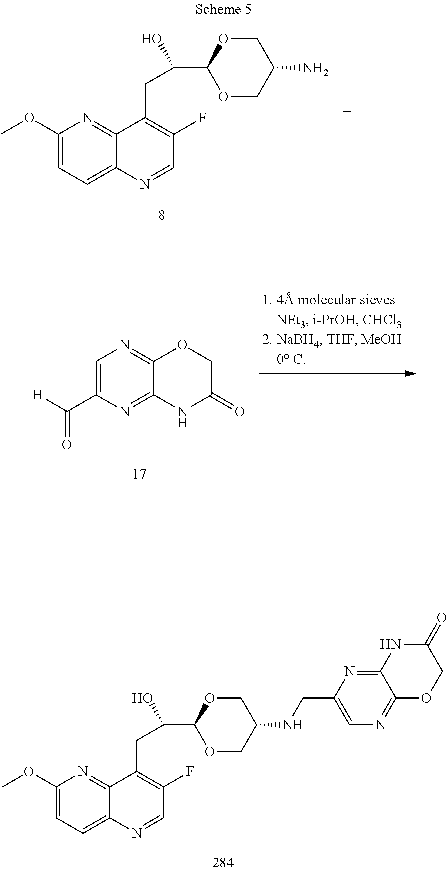 [SMILES: COc1ccc2ncc(F)c(C[C@H](O)[C@H]3OC[C@H](N)CO3)c2n1 ; O=Cc1cnc2c(n1)NC(=O)CO2 ; COc1ccc2ncc(F)c(C[C@H](O)[C@H]3OC[C@H](NCc4cnc5c(n4)NC(=O)CO5)CO3)c2n1]

### 6-(((2-((S)-2-(3-fluoro-6-methoxy-1,5-naphthyridin-4-yl)-1-hydroxyethyl)-trans-1,3-dioxan-5-yl)amino)methyl)-2H-pyrazino[2,3-b][1,4]oxazin-3(4H)-one (284)

A flask containing (1S)-2-(3-fluoro-6-methoxy-1,5-naphthyridin-4-yl)-1-[5-amino-trans-1,3-dioxan-2-yl]ethan-1-ol (8, 650 mg, 2.01 mmol, 1 equiv), 3-oxo-2H,3H,4H-pyrazino[2,3-b][1,4]oxazine-6-carbaldehyde (17, 414 mg, 2.31 mmol, 1.15 equiv) and 4 Å molecular sieves (300 mg/mmol of amine) was nitrogen and vacuum purged prior to the addition of anhydrous CHCl₃ (43 mL) and i-PrOH (13 mL) by syringe. Triethylamine (378 μL, 2.71 mmol, 1.35 equiv) was added dropwise by syringe. The reaction was stirred at room temperature for 48 hours. The reaction mixture was concentrated. The flask was equipped with a nitrogen line and the dried material was taken up in anhydrous THF (12.7 mL) and anhydrous MeOH (15.8 mL), both added by syringe. The solution was stirred at 0° C. for 30 minutes at which point NaBH₄ (38 mg,l mmol, 0.5 equiv) was added portion wise. The reaction continued to stir at 0° C. for 1.5 hours and a second addition of NaBH₄ (12 mg, 0.32 mmol, 0.2 equiv) was added to the reaction mixture and stirred at 0° C. for 15 minutes. The reaction was quenched with saturated aqueous NaHCO₃ and transferred into a separatory funnel where it was diluted with H₂O and DCM. The phases were separated, and the aqueous layer was extracted with DCM (50 mL×3) followed by 5% MeOH in DCM (50 mL×4). The combined organic layers were dried with Na₂SO₄, decanted, and concentrated to afford an off white solid. The material was loaded onto silica gel and purified by flash column chromatography on silica gel (MeOH/DCM. 0 to 2.5% gradient over 40 minutes). Product eluted in ~0.5% MeOH and was repurified by flash column chromatography on silica gel (MeOH/DCM 0 to 5% gradient over 30 minutes). The title compound was a white solid (300 mg, 0.62 mmol, 31%). LRMS [M+H]⁻=487.31. ¹H NMR (DMSO-d₆) δ: 11.55 (br s, 1H): 8.73 (s, 1H): 8.26 (d, J=9.0 Hz, 1H): 7.78 (s, 1H); 7.21 (d, J=9.0 Hz, 1H); 4.86 (s, 2H), 4.83 (d, J=6.0 Hz, 1H); 4.39 (d, J=4.2 Hz, 1H); 4.16-4.08 (m. 2H); 4.02 (s, 3H); 4.01-3.95 (m, 1H); 3.72 (br d, J=5.9 Hz, 2H); 3.40-3.34 (m, 1H); 3.31-3.24 (m, 2H, partially obscured by water); 3.17-3.11 (m, 1H); 2.83-2.72 (m, 1H); 2.14-2.06 (m, 1H).

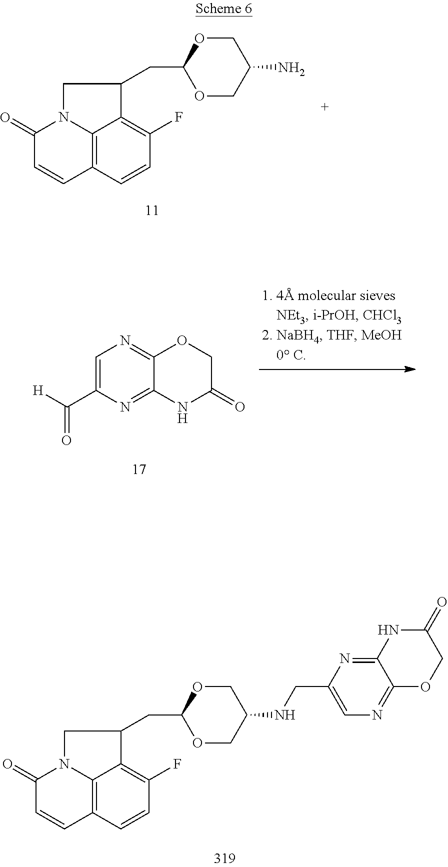 [SMILES: N[C@H]1CO[C@H](CC2Cn3c(=O)ccc4ccc(F)c2c43)OC1 ; O=Cc1cnc2c(n1)NC(=O)CO2 ; O=C1COc2ncc(CN[C@H]3CO[C@H](CC4Cn5c(=O)ccc6ccc(F)c4c65)OC3)nc2N1]

### 6-(((2-((9-fluoro-4-oxo-1,2-dihydro-4H-pyrrolo[3,2,1-ij]quinolin-1-yl)methyl)-trans-1,3-dioxan-5-yl)amino)methyl)-2H-pyrazino[2,3-b][1,4]oxazin-3(4H)-one (319)

A flask containing 1-((5-amino-trans-1,3-dioxan-2-yl)methyl)-9-fluoro-1,2-dihydro-4H-pyrrolo[3,2,1-ij]quinolin-4-one (11, 100 mg, 0.329 mmol, 0.78 equiv), 3-oxo-2H,3H,4H-pyrazino[2,3-b][1,4]oxazine-6-carbaldehyde (17, 76 mg, 0.42 mmol, 1 equiv) and 4A molecular sieves (300 mg/mmol of amine) was nitrogen and vacuum purged 3 times. To the flask was added anhydrous i-PrOH (2.75 mL) and anhydrous CHCl₃ (9.13 mL). Triethylamine (0.08 mL, 0.572 mmol, 1.35 equiv) was added to the stirring solution by syringe. The reaction was stirred under nitrogen for 48 hours to ensure imine formation. The reaction mixture was concentrated and taken up in anhydrous THF (2.1 mL) and anhydrous MeOH (2.6 mL). The reaction mixture was stirred at 0° C. for 30 minutes prior to the addition of NaBH₄ (6 mg, 0.16 mmol. 0.5 equiv). The mixture was stirred for 45 minutes and was quenched with saturated NaHCO₃ (1.5 mL). The mixture was diluted with DCM and H₂O. The phases were separated, and the aqueous layer was extracted with DCM (20 mL×4) followed by a 2% MeOH in DCM solution (30 mL x1). The combined organic layers were washed with brine, dried with Na₂SO₄, decanted, and concentrated to afford a light-yellow solid. The crude product was purified by flash chromatography on silica gel (DCM/MeOH 0 to 2% gradient over 40 minutes). The title compound eluted in 0.4% MeOH. Concentration in vacuo afford the title compound as an off white solid (58 mg, 0.12, 29%). LRMS [M+H]⁺=468.33. ¹H NMR (DMSO-d₆) δ: 11.53 (br s, 1H); 7.88 (d, J=9.4 Hz, 1H); 7.78 (s, 1H): 7.58 (dd, J=8.6, 4.6 Hz, 1H): 7.00 (dd. J=9.7, 8.9 Hz, 1H): 6.50 (d, J=9.4 Hz, 1H); 4.86 (s, 2H); 4.63 (t, J=4.8 Hz, 1H); 4.40 (dd, J=12.7, 9.5 Hz, 1H); 4.19-4.06 (m, 3H); 4.04-3.94 (m, 1H); 3.72 (s, 2H); 3.34-3.24 (m, 2H, partially obscured by water); 2.86-2.74 (m, 1H); 2.25-2.07 (m, 2H); 1.99-1.89 (m, 1H).

Compound 284 has also been prepared according to the procedures described below:

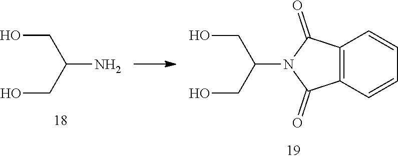 [SMILES: NC(CO)CO ; O=C1c2ccccc2C(=O)N1C(CO)CO]

### 2-(1,3-dihydroxypropan-2-yl)isoindoline-1,3-dione (19)

To a 3-neck, 500-mL RBF was added serinol (18, 10 g, 110 mmol, 1.0 equiv), an oven-dried stir bar, followed by 1-butanol (110 mL). Set to stir as reaction mixture was heated to an internal temperature of 120° C. via heating mantle (monitored and powered by J-KEM temperature controller). Once solution was homogenous (~40° C.), phthalic anhydride (16.3 g, 110 mmol, 1.0 equiv) was added in 5 portions with continued heating, allowing 10° C. increases between additions. Middle neck was fitted w/a Dean-Stark apparatus. As mixture came up to reflux, it was homogenous w/a slightly yellow tinge to it; flask insulated w/aluminum foil to allow vapor to carry into the Dean-Stark apparatus. After stirring for ~12 hours at reflux, a small aliquot was removed and concentrated for evaluative NMR analysis, which showed incomplete reaction. After stirring for 18 hours at reflux, reaction was cooled to an internal temperature of 50° C., then 110 mL of MTBE was added. Upon addition, a large amount of precipitate formed. Large chunks were manually broken up, then 220 mL of hexanes was added. Submerged in ice-water bath and allowed to stir for 30 additional minutes. Crude product was then isolated via vacuum filtration on a Buchner funnel, rinsing flask (and filter cake) with 110 mL of hexanes. Allowed to dry on diaphragm pump for a few minutes, then transferred solid to a 500-mL RBF and dried on rotary evaporator (50° C. 25 mbar). Subsequent drying under high vacuum afforded the title compound (21.712 g, 81.0%) as a white powder. The crude product dissolves with heating in H₂O (4 mL/g) and affords white needles upon gradual cooling to ambient temperature followed by overnight storage at ~10° C. Collection by vacuum filtration and rigorous drying under vacuum affords the crystalline product.

¹H NMR (DMSO-d₆, 400 MHz): δ 7.89-7.80 (m, 4H), 4.85 (dd, J=6.5, 5.5 Hz, 2H), 4.24 (tt, J=8.8, 5.6 Hz, 1H), 3.80 (ddd, J=11.1, 8.9, 5.5 Hz, 2H), 3.72-3.59 (m, 2H).

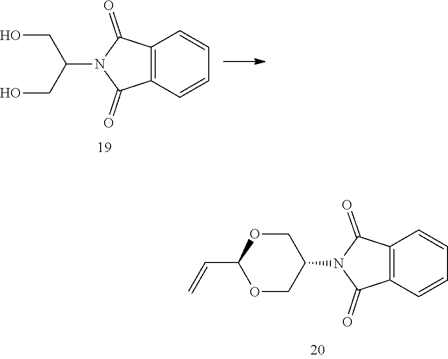 [SMILES: O=C1c2ccccc2C(=O)N1C(CO)CO ; C=C[C@H]1OC[C@H](N2C(=O)c3ccccc3C2=O)CO1]

### 2-(2-vinyl-trans-1,3-dioxan-5-yl)isoindoline-1,3-dione (20)

To an oven-dried, 1-L, 3-neck RBF was added a magnetic stir bar. Flask put under N₂ atmosphere (3 cycles of evacuate and backfill), then charged with 2-(1,3-dihydroxypropan-2-yl)isoindoline-1,3-dione (19, 13.8 g, 62.2 mmol, 1.0 equiv), DCM (311 mL), and 42 grams of oven-dried 5A molecular sieves. Acrolein dimethyl acetal (6.67 g, 7.76 mL, 65.3 mmol, 1.05 equiv) was then added via graduated cylinder (rinsed with 3 mL of DCM), followed by dropwise addition of BF₃·Et₂O (441 mg, 0.384 mL, 3.11 mmol, 0.05 equiv) across 1-2 minutes. Allowed to vigorously stir at RT. After stirring for 3 hours, reaction was quenched by addition of triethylamine (629 mg, 0.867 mL, 6.22 mmol, 0.1 equiv), then filtered through a thin pad of Celite in a fritted glass funnel, rinsing reaction flask and filter cake w/DCM until UV analysis showed no more product eluting. Receiving flask charged w/64 mL of MeOH then DCM was removed on rotary evaporator. Cooled to RT and allowed product to precipitate, then isolated via vacuum filtration on a Buchner funnel, rinsing with small amounts of cold MeOH. A second crop was taken from the filtrate by removing most of the MeOH under vacuum and repeating the process. Drying the isolated solids under vacuum afforded the title compound (10.56 g, 65.5%) as a cream-colored solid.

¹H NMR (CDCl₃, 400 MHz), δ 7.89-7.80 (m, 2H), 7.78-7.69 (m, 2H), 5.90 (ddd, J=17.4, 10.6, 4.5 Hz, 1H), 5.53 (dt, J=17.5, 1.2 Hz, 1H), 5.35 (dt, J=10.6 Hz, 1.1 Hz, 1H), 5.10 (app d, J=4.6 Hz, 1H), 4.72-4.60 (m. 1H), 4.57-4.45 (m, 2H), 4.14-4.04 (m, 2H).

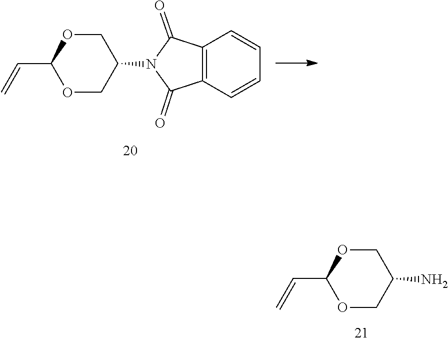

### 2-vinyl-trans-1,3-dioxan-5-amine (21)

To a 3-neck, 250-mL RBF was added 2-(2-vinyl-trans-1,3-dioxan-5-yl)isoindoline-1,3-dione (20, 10.6 g, 40.7 mmol, 1.0 equiv) and a magnetic stir bar. Suspended in EtOH (102 mL) and set to stir at RT as ethylenediamine (7.34 g, 8.16 mL, 122 mmol, 3 equiv) was added in one portion at RT. After a few minutes of stirring, reaction mixture had largely homogenized. Middle neck fitted with waterless reflux condenser, right neck capped w/rubber septum, left neck capped w/rubber septum that had been pierced with temperature probe. Heated to reflux via heating mantle (monitored and powered by J-KEM temperature controller). After stirring overnight at reflux, a large amount of white precipitate had formed, and reaction mixture had a paint-like consistency and appearance. Cooled to ambient temperature by submerging flask in a water bath, then added 100 mL (1 vol.) of DCM. Once at ambient temperature, mixture was cooled further by then submerging in an ice-water bath. Filtered through Celite, rinsing sides of reaction flask w/a small amount of DCM. After all of mixture had been filtered, filter cake rinsed w/1 volume of DCM. TLC (19/1 DCM/MeOH, stain w/DNP) of last eluent still showed desired product in the filter cake. Filtrate concentrated under vacuum, then reattached to fritted funnel and eluted filter cake with 200 mL of MeOH. Combined filtrates were then concentrated onto 15 grams of silica gel and loaded onto a short column of silica (60 grams, packed w/DCM) in a 350-mL fritted funnel, top layer of silica protected with filter paper. Eluted with 150-mL fractions of 100% DCM, 2.5% MeOH in DCM, then 5% MeOH in DCM (end of gradient). TLC analysis showed that the last of the product eluted in the 2nd 5% fraction. Concentration of product fractions (including an azeotropic drying with toluene) in a pre-weighed, 500-mL RBF afforded the title compound (4.74 g, 4.503 g adjusting for toluene contamination, 85.6%) as a yellow oil. NMR shows desired product in good purity, but some toluene remains (~5:1 molar ratio of desired to toluene, ~95 wt % desired). Taken into next step without further purification.

¹H NMR (CDCl₃, 400 MHz): δ 5.86 (ddd, J 17.4, 10.6, 4.4 Hz, 1H), 5.48 (dt, J=17.5, 1.2 Hz, 1H), 5.31 (dt, J=10.6 Hz, 1.2 Hz, 1H), 4.85 (app d, J=4.4 Hz, 1H), 4.24-4.13 (m, 2H), 3.36-3.27 (m, 2H), 3.11 (tt, J=10.4, 4.9 Hz, 1H), 1.31 (br s, 2H).

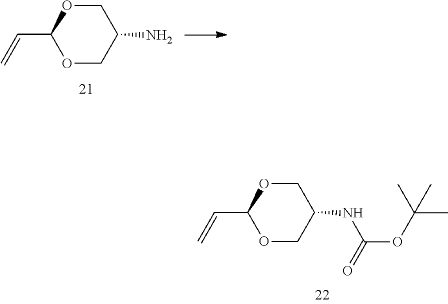

### tert-butyl (2-vinyl-trans-1,3-dioxan-5-yl)carbamate (22)

To a 500-mL RBF containing neat 2-vinyl-trans-1,3-dioxan-5-amine (21, 4.74 g, 95 wt %, 36.7 mmol, 1.0 equiv) was added a magnetic stir bar then DCM (34 mL). Set to stir at RT as liquid di-tert-butyl dicarbonate (8.01 g, 8.43 mL, 36.7 mmol, 1.0 equiv) was added in one portion via syringe; gas evolution started within 15 seconds of addition. Allowed to stir at RT for 30 minutes, then charged flask with heptane (33 mL). Removed DCM on rotavap (48° C., 700 mbar). This gave a slurry of a fluffy white solid in heptane. Stirred in an ice bath for a few minutes, then isolated via vacuum filtration on a Buchner funnel. The filtrate was placed in freezer overnight, and a second crop of solid was combined with the earlier material to afford the title compound (6.626 g, 83%) as a fluffy white solid after azeotropic drying from toluene.

¹H NMR (CDCl₃, 400 MHz): δ 5.86 (ddd, J=17.4, 10.7, 4.4 Hz, 1H), 5.48 (dt, J=17.5, 1.3 Hz, 1H), 5.33 (dt, J=10.8, 1.3 Hz, 1H), 4.87 (app d, J=4.4 Hz, 1H), 4.28-4.21 (m, 2H), 3.90 (br s, 1H), 3.40 (app t, J=10.4 Hz, 2H), 1.44 (s, 9H).

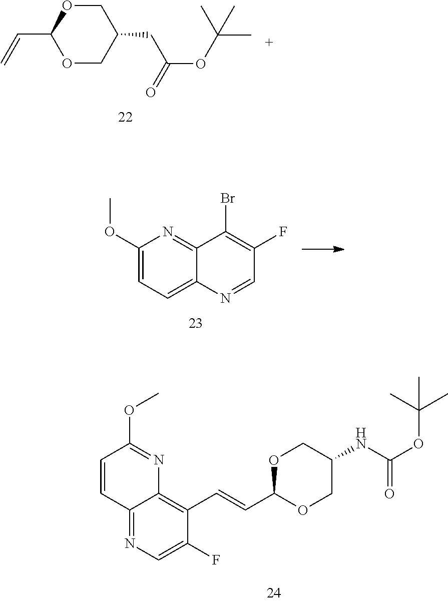 [SMILES: C=C[C@H]1OC[C@H](CC(=O)OC(C)(C)C)CO1 ; COc1ccc2ncc(F)c(Br)c2n1 ; COc1ccc2ncc(F)c(/C=C/[C@H]3OC[C@H](NC(=O)OC(C)(C)C)CO3)c2n1]

### tert-butyl (2-((E)-2-(3-fluoro-6-methoxy-1,5-naphthyridin-4-yl)vinyl)-trans-1,3-dioxan-5-yl)carbamate (24)

To an oven-dried, 3-neck, 250-mL RBF was added an oven-dried stir bar, tert-butyl (2-vinyl-trans-1,3-dioxan-5-yl)carbamate (22, 6.63 g, 28.9 mmol, 1.1 equiv), palladium acetate (118 mg, 0.53 mmol, 0.02 equiv). 8-bromo-7-fluoro-2-methoxy-1,5-naphthyridine (23, 6.75 g, 26.3 mmol, 1 equiv), and potassium bicarbonate (13.2 g, 131 mmol, 5 equiv). Necks fitted with rubber septa; left neck pierced with temperature probe. Flask put under N₂ atmosphere (3 cycles of evacuate and backfill), then charged with 52.5 mL of anhydrous DMF (from solvent purification system, then dried over activated 3A sieves for ~6 hours). Set to stir as reaction was heated to an internal temperature of 80° C. via heating mantle (monitored and powered by J-KEM temperature controller). After stirring for ~10.5 hours at 80° C., TLC (1/1 Hex/EA) shows a small amount of SM, a large amount of desired product and some undesired ester side product. Cooled in a water bath and diluted with 105 mL of EA and filtered through Celite, rinsed flask with 53 mL of EA. UV analysis showed desired product remained in cake or frit. Rinsed filter cake with ~200 mL of EA. Filtrate concentrated under vacuum then suspended the resulting brown paste in 53 mL of MTBE. Isolated precipitate via vacuum filtration, rinsing flask and solid with MTBE. Allowed to dry on diaphragm pump for a few minutes, then dried under vacuum. A second crop of product was isolated from the MTBE filtrate by concentrating under vacuum, precipitating again from a smaller volume of MTBE, and filtering. A third crop of product was then isolated by taking the original Celite filter cake and suspending it in DCM (enough volume to cover). This suspension was stirred at RT for a few minutes, then once again filtered in a fritted glass funnel to give a pale-yellow filtrate. NMR analysis of this third crop shows it to be in sufficient purity to combine with the first two crops. Concentration of the pale-yellow filtrate and combining with the other two isolates afforded the title product (7.83 g, 73.5%) as a fluffy tan solid.

¹H NMR (CDCl₃, 400 MHz): δ 8.66 (d, J=2.1 Hz, 1H), 8.17 (d, J=9.0 Hz, 1H), 7.61 (dd, J=16.7, 1.0 Hz, 1H), 7.17 (dd, J=16.7, 4.4 Hz, 1H), 7.09 (d, J=9.0 Hz, 1H), 5.19 (br d, J=4.0 Hz, 1H), 4.39-4.30 (m, 2H), 4.11 (s, 3H), 3.97 (br s, 1H), 3.52 (app t, J=10.4 Hz, 2H), 1.46 (s, 9H).

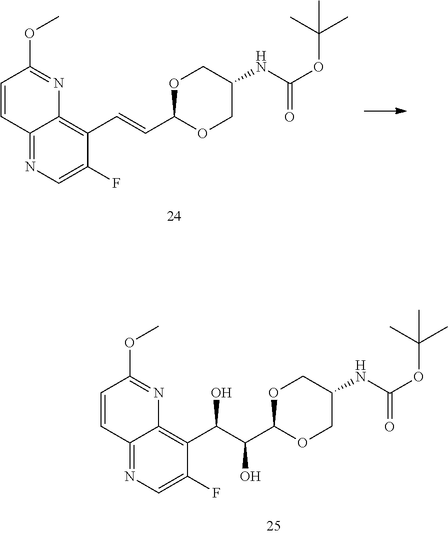 [SMILES: COc1ccc2ncc(F)c(/C=C/[C@H]3OC[C@H](NC(=O)OC(C)(C)C)CO3)c2n1 ; COc1ccc2ncc(F)c([C@@H](O)[C@H](O)[C@H]3OC[C@H](NC(=O)OC(C)(C)C)CO3)c2n1]

### tert-butyl (2-((1S,2R)-2-(3-fluoro-6-methoxy-1,5-naphthyridin-4-yl)-1,2-dihydroxyethyl)-trans-1,3-dioxan-5-yl)carbamate (25)

To a 1-L RBF was added methanesulfonamide (1.84 g. 19.3 mmol, 1 equiv), followed by t-BuOH and water (193 mL of each). Set to stir at RT as AD-mix beta (77.2 g) was added in portions to give a viscous, deep red opaque mixture. Flask submerged in an ice-water bath and allowed to stir until internal temperature approached 0° C. (~15 minutes of cooling), tert-butyl (2-((E)-2-(3-fluoro-6-methoxy-1,5-naphthyridin-4-yl)vinyl)-trans-1,3-dioxan-5-yl)carbamate (24, 7.83 g. 19.3 mmol. 1 equiv) was then added to the cooled mixture, rinsing container with 19.3 mL of DCM that was then added to the reaction mixture. After stirring for 16.5 hours (gradually warming to RT overnight), TLC (4/1 EA/Hex) shows a trace amount of SM with a large amount of desired product. After stirring for 18 hours, reaction was quenched by addition of 83 grams of sodium sulfite, followed by 193 mL of water. After stirring for 15-20 minutes, mixture was transferred to a separatory funnel (flask rinsed w/small amount of water) and layers were separated. Aqueous phase was then extracted once w/100 mL of EA. Combined organic layers washed with 100 mL of water, then 100 mL of 1M NaOH_{aq}, then 100 mL of brine. Organic extract dried over sodium sulfate then concentrated on rotary evaporator to afford a pink foam that was scraped to release into a soft solid. Further drying under high vacuum gave 9.86 grams of crude pink product. Dissolved in DCM and concentrated onto 27 grams of silica gel, then loaded onto a short column of silica (108 grams, packed w/4/1 EA/Hex) in a 350-mL fritted funnel. Elution w/1.25 L of 4/1 Hex/EA fully removed the desired product from the column (confirmed by UV analysis on TLC plate). Concentration of eluent afforded the title compound (7.31 g, 86.1%) as a light tan foam that was manually broken up to give a glassy solid. NMR shows desired product in high purity with some EA (2.4:1 ratio, 92.3 wt % purity).

¹H NMR (CDCl₃, 400 MHz): δ 8.66 (d, J=0.5 Hz, 1H), 8.25 (d, J=9.2 Hz, 1H), 7.12 (d, J=9.2 Hz, 1H), 5.65 (d, J=3.8 Hz, 1H), 4.64 (d, J=5.1 Hz, 1H), 4.28-4.20 (m, 2H), 4.06 (s, 3H), 3.97 (t, J=4.6 Hz, 1H), 3.92 (br s, overlaps with other peak, 1H), 3.35 (t, J=10.8 Hz, overlaps with other peak, 1H), 3.33 (t, J=10.8 Hz, 1H), 1.43 (s, 9H).

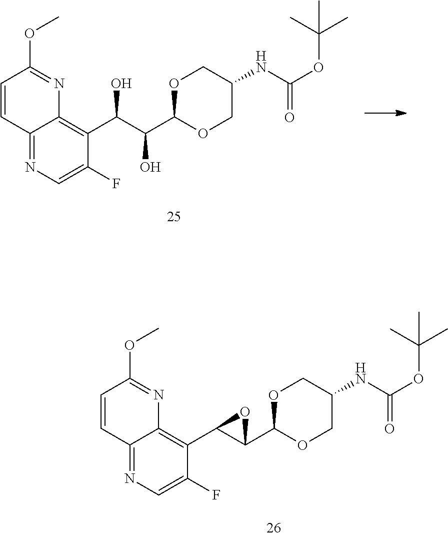 [SMILES: COc1ccc2ncc(F)c([C@@H](O)[C@H](O)[C@H]3OC[C@H](NC(=O)OC(C)(C)C)CO3)c2n1 ; COc1ccc2ncc(F)c([C@H]3O[C@@H]3[C@H]3OC[C@H](NC(=O)OC(C)(C)C)CO3)c2n1]

### tert-butyl (2-((2S,3R)-3-(3-fluoro-6-methoxy-1,5-naphthyridin-4-yl)oxiran-2-yl)-trans-1,3-dioxan-5-yl)carbamate (26)

To a 250-mL RBF was added tert-butyl (2-((1S,2R)-2-(3-fluoro-6-methoxy-1,5-naphthyridin-4-yl)-1,2-dihydroxyethyl)-trans-1,3-dioxan-5-yl)carbamate (25, 7.31 g, 16.6 mmol, 1.0 equiv) and a magnetic stir bar. Dissolved in 73 mL of DCM with stirring as PPTS (209 mg, 0.83 mmol, 0.05 equiv) was added to the bronze-orange solution. Once dissolved, trimethyl orthoacetate (2.99 g. 3.38 mL. 24.9 mmol, 1.5 equiv) was added in one portion via syringe. After 2 hours, stir bar was removed and rinsed with a small amount of DCM, then reaction mixture was concentrated on rotary evaporator (50° C., 700 mbar for 7 minutes, then gradually brought down to 50 mbar), followed by careful drying under high vacuum for a few minutes to remove trace MeOH. The syrup readily foamed under high vacuum, but eventually solidified. This was manually broken up with a spatula, then dissolved in 73 mL of DCM. Set to stir as flask was submerged in an ice-water bath; flask was charged with a catalytic amount of MeOH (2 drops from a 26-gauge needle), followed by addition of TMSCl (2.71 g, 3.16 mL, 24.9 mmol, 1.5 equiv) across ~2 minutes. Allowed to stir in ice bath for 5 minutes, then removed and allowed to warm to RT. After stirring for 2 hours, reaction mixture was gently concentrated under vacuum as before. Taken up in 73 mL of MeOH to give a cloudy, orange mixture. Set to stir at RT then charged flask with potassium carbonate (6.89 g, 49.8 mmol, 2.0 equiv). Upon addition, mixture quickly turned from orange to pink/purple. After stirring for 10 minutes, TLC shows full consumption of acetoxy chloride intermediate, and reaction mixture significantly thickened and became lighter due to the generation of potassium chloride. Mixture poured into 73 mL of a half-saturated ammonium chloride/ice slurry. Manually swirled for a minute or two to dissolve salts and melt ice, then isolated precipitate via vacuum filtration on a Buchner funnel, rinsing reaction flask and precipitate with water; this gave a whitish isolate and a pink filtrate. After drying for a few minutes on diaphragm pump, isolate (wet clay texture) was transferred to a 125-mL Erlenmeyer flask. This was capped with a rubber septum (pierced with a needle) and wrapped with Parafilm, then dried in a vacuum desiccator (with Drierite) under high vacuum for 9 hours. This afforded a solid that was clearly drier than before, but not completely dry (9.69 grams, 138% of theoretical). Broke up chunks to give a solid that was handleable but was still visually wet. Dried on rotary evaporator (52° C., 200 mbar brought down to maximum vacuum). After drying for ~15-20 minutes on rotary evaporator, briefly dried under high vacuum to afford the title compound (6.13 g, 87.6%) as a free-flowing, eggshell-white solid. NMR shows desired product in high purity with only a small amount of water.

¹H NMR (CDCl₃, 400 MHz): δ 8.62 (d, J=1.8 Hz, 1H), 8.18 (d, J=9.1 Hz, 1H), 7.10 (d, J=9.0 Hz, 1H), 4.81 (d, J=2.1 Hz, 1H), 4.66 (d, J=3.5 Hz, 1H), 4.36-4.27 (m, 2H), 4.23 (br s, overlaps with other peaks, 1H), 4.11 (s, 3H), 4.04 (dd, J=3.5, 2.3 Hz, 1H), 3.97 (br s, overlaps with other peak, 1H), 3.49-3.37 (m, 2H), 1.45 (s, 9H).

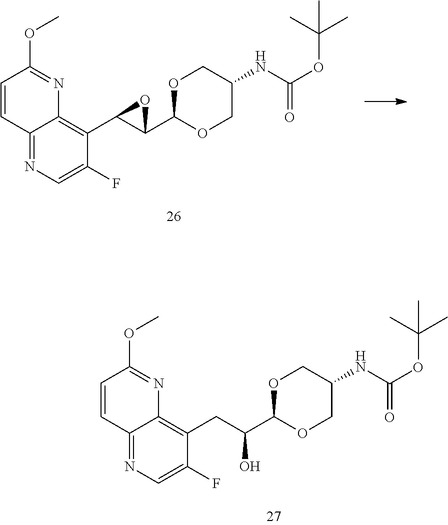 [SMILES: COc1ccc2ncc(F)c([C@H]3O[C@@H]3[C@H]3OC[C@H](NC(=O)OC(C)(C)C)CO3)c2n1 ; COc1ccc2ncc(F)c(C[C@H](O)[C@H]3OC[C@H](NC(=O)OC(C)(C)C)CO3)c2n1]

### tert-butyl (2-((S)-2-(3-fluoro-6-methoxy-1,5-naphthyridin-4-yl)-1-hydroxyethyl)-trans-1,3-dioxan-5-yl)carbamate (27)

To a 3-neck, 250-mL RBF was added ammonium formate (3.66 g, 58.1 mmol, 4 equiv) and a magnetic stir bar. Added 97 mL of MeOH and set to stir until all ammonium formate had dissolved, then equipped middle neck of flask with a waterless reflux condenser and fitted left and right necks with rubber septa (left neck pierced with temperature probe), tert-butyl (2-((2S,3R)-3-(3-fluoro-6-methoxy-1,5-naphthyridin-4-yl)oxiran-2-yl)-trans-1,3-dioxan-5-yl)carbamate (26, 6.12 g. 14.5 mmol, 1 equiv) was added to the solution to give a creamy, heterogenous mixture. Set to heat to an internal temperature of 60° C. via heating mantle (monitored and powered by J-KEM temperature controller). At an internal temperature of 40° C., 10% Pd/C (55% wetted with water, 1.22 g) was added (rinsing sides of flask and funnel w/a small amount of MeOH). At an internal temperature of 45° C., mild gas evolution was observed; between 53-54° C. most of the epoxide had gone into solution (noted due to overall darkening of color and no more observable pellets of substrate). Mild reflux observed at 58° C., TLC (6/4 EA/Hex) shows a faint amount of SM and a large amount of desired product. Allowed to stir for 15 minutes at reflux, then cooled to ambient temperature in a water bath. Filtered through a pad of Celite, rinsing the flask and filter cake w/MeOH until no more desired product eluted through the filter. To the golden filtrate was added 60 mL of water to give some cloudiness. MeOH was removed on rotary evaporator (150 mbar, 51° C.) which gave a syrupy mass in the water layer. Cooled flask in an ice-water bath and vigorously stirred the mass to triturate out desired product. Isolated product via vacuum filtration with a Buchner funnel, rinsing the flask and solid with water. Thorough drying on diaphragm pump and under vacuum afforded the title compound (5.784 g. 94.1%) as a whitish solid. NMR shows desired product in high purity, small amount of formate and MeOH remain.

¹H NMR (CDCl₃, 400 MHz): δ 8.65 (s, 1H), 8.21 (d, J=9.1 Hz, 1H), 7.08 (d, J=9.0 Hz, 1H), 4.45 (d, J=3.9 Hz, 1H), 4.32-4.22 (m, 2H), 4.17 (d, J=8.6 Hz, overlaps with other peaks, 1H), 4.13-4.04 (m, 1H, overlaps with —OCH₃ peak, 1H), 4.08 (s, 3H), 3.95 (br s, 1H), 3.53 (app ddd, J=13.5, 3.4, 1.2 Hz, 1H), 3.41 (app ddd, J=13.5, 8.6, 1.1 Hz, overlaps with other peaks, 1H), 3.35 (t, J=10.6 Hz, overlaps with other peaks, 1H), 3.33 (t, J=10.7 Hz, overlaps with other peaks, 1H), 1.44 (s, 9H).

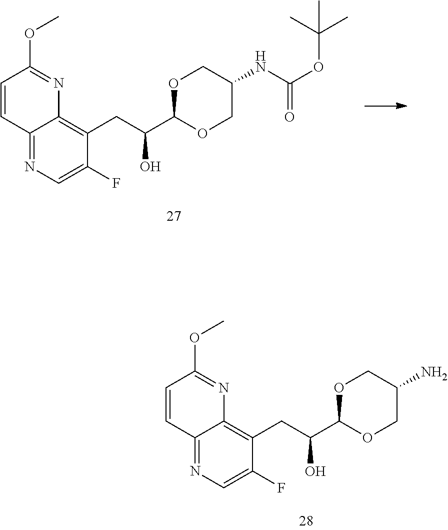 [SMILES: COc1ccc2ncc(F)c(C[C@H](O)[C@H]3OC[C@H](NC(=O)OC(C)(C)C)CO3)c2n1 ; COc1ccc2ncc(F)c(C[C@H](O)[C@H]3OC[C@H](N)CO3)c2n1]

### (S)-1-(5-amino-trans-1,3-dioxan-2-yl)-2-(3-fluoro-6-methoxy-1,5-naphthyridin-4-yl)ethan-1-ol (28)

An oven-dried, 250-mL RBF was put under N₂ atmosphere (3 cycles of evacuate and backfill), then charged with anhydrous 4M HCl in dioxane (34.1 mL, 137 mmol, 10 equiv). Set to stir at RT as septum was briefly removed and tert-butyl (2-((S)-2-(3-fluoro-6-methoxy-1,5-naphthyridin-4-yl)-1-hydroxyethyl)-trans-1,3-dioxan-5-yl)carbamate (27, 5.78 g, 13.7 mmol. 1 equiv) was added portion-wise across a few minutes. Upon addition, the mixture took on a yellow color (with some green overtones), a syrupy mass precipitated out, gas evolution was observed, and a modest exotherm occurred. Septum replaced and allowed to stir under a stream of nitrogen. After stirring for 10 minutes, TLC (100% EA) shows no remaining SM and everything on baseline, no more gas evolution. Allowed to stir for another 10 minutes to ensure full reactivity, then concentrated gently on rotary evaporator (50° C., 400 mbar gradually brought down to 100 mbar over 5 minutes); pulled maximum vacuum after 100 mbar had been applied for 10 minutes. The resulting foam/gum was then dried twice from EtOH (50 mL per concentration), followed by a final azeotropic drying from toluene (50 mL). Further drying under high vacuum afforded quantitative recovery of the unprotected hydrochloride salt with the appearance of light brown crumbs. NMR shows desired product with a considerable amount of EtOH remaining and trace toluene. Hydrochloride salt taken up in MeOH (68 mL) and set to stir as flask was submerged in an ice-water bath. To the cooled, nearly heterogenous mixture was then added triethylamine (4.15 g, 5.71 mL, 41 mmol, 3 equiv), dropwise, across ~2 minutes. Upon addition, a layer of vapor formed above the liquid layer, and the reaction mixture darkened to a medium brown. After stirring for ~5 minutes, MeOH was removed on rotary evaporator to afford a pasty solid. Co-evaporation from 50 mL of MTBE gave a more solid mixture. Suspended in 100 mL of THF and set to vigorously stir to break up large chunks and dissolve as much desired product as possible; suspension gave a golden supernatant with a powdery white precipitate that settled to the bottom of the flask. Mixture filtered through a short pad of Celite, rinsing the flask and filter cake with THF until UV analysis shows no more desired product eluting. Cloudy, golden filtrate concentrated under vacuum (became transparent when warmed in rotavap bath) to afford a pasty solid. Scraping from MTBE, then subsequent evaporation afforded the title compound (3.555 g, 72.3%) as a light yellow solid. NMR shows desired product in good purity, with minor baseline impurities and trace MTBE.

¹H NMR (CD₃OD, 400 MHz): δ 8.61 (d, J=1.0 Hz, 1H), 8.19 (d, J=9.1 Hz, 1H), 7.16 (d, J=9.1 Hz, 1H), 4.49 (d, J=4.0 Hz, 1H), 4.22-4.12 (m, 3H), 4.10 (s, 3H), 3.78-3.63 (m, 1H), 3.61-3.52 (m, 1H), 3.42-3.29 (m, 3H, overlaps with solvent peak), 3.09-2.98 (m, 1H).

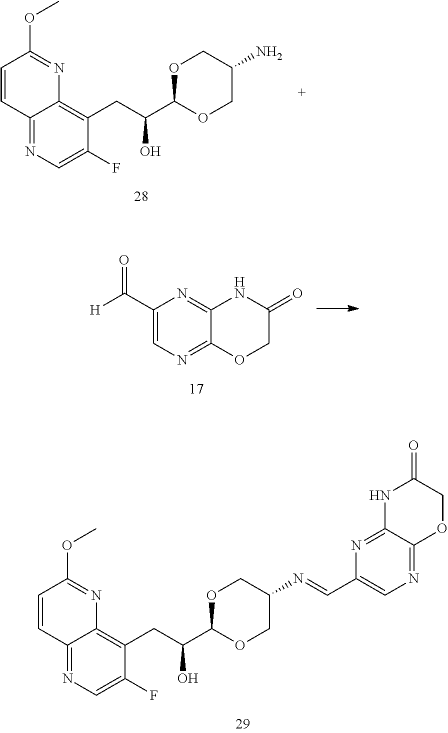 [SMILES: COc1ccc2ncc(F)c(C[C@H](O)[C@H]3OC[C@H](N)CO3)c2n1 ; O=Cc1cnc2c(n1)NC(=O)CO2 ; COc1ccc2ncc(F)c(C[C@H](O)[C@H]3OC[C@H](/N=C/c4cnc5c(n4)NC(=O)CO5)CO3)c2n1]

### 6-((E)-((2-((S)-2-(3-fluoro-6-methoxy-1,5-naphthyridin-4-yl)-1-hydroxyethyl)-trans-1,3-dioxan-5-yl)imino)methyl)-2H-pyrazino[2,3-b][1,4]oxazin-3(4H)-one (29)

To a 250-mL RBF was added (S)-1-(5-amino-trans-1,3-dioxan-2-yl)-2-(3-fluoro-6-methoxy-1,5-naphthyridin-4-yl)ethan-1-ol (28, 2.30 g, 7.13 mmol, 1 equiv), a magnetic stir bar, and 71 mL of IPA. Briefly warmed and sonicated to dissolve most of the large chunks, then set to vigorously stir at RT as 3-oxo-2H,3H4H-pyrazino[2,3-b][1,4]oxazine-6-carbaldehyde (17, 1.28 g, 7.13 mmol, 1 equiv) was added in one portion. After stirring for ~15 minutes, TLC shows a very minor amount of imine formation. Gently concentrated on rotavap (50° C., 400 mbar brought down to 100 mbar, then maximum vacuum) to afford a tannish pasty solid. Resuspended in the same volume of IPA and set to stir at RT, TLC now shows imine as the major component with small amounts of SM remaining. After stirring for another ~30 minutes, concentrated as before to give a more granular solid. This cycle was repeated one more time. NMR of the new solid shows desired product as major component, but some aldehyde and other components are present. Tried to suspend and filter with IPA. Drying gave a yellow solid; NMR shows one of the unknown components is gone and a 4:1 ratio of desired product to aldehyde SM. Repeated above process with ~25 mL of THF to give desired product in reasonably good purity (~0.04 dioxane-containing compound, ~0.03 aldehyde). After the combined filtrates stood overnight in the hood, a large amount of precipitate had crashed out of solution and looked nearly colorless with a yellow supernatant. This was isolated via vacuum filtration and combined with the original isolate and dried under vacuum. NMR shows desired product in high purity with considerable amounts of THF and IPA remaining. Further drying under high vacuum afforded the title compound (2.243 g. 65.0%) as a granular, light yellow solid.

¹H NMR (DMSO-d₆, 400 MHz): δ 11.80 (s, 1H), 8.75 (s, 1H), 8.41 (s, 1H), 8.28 (d, J=9.0 Hz, 1H), 8.18 (s, 1H), 7.23 (d, J=9.0 Hz, 1H), 4.94 (s, 2H), 4.60 (d, J=4.2 Hz, 1H), 4.14-3.97 (m, overlaps with —OCH₃ peak, 4H), 4.04 (s, 3H), 3.83-3.69 (m, 3H), 3.49-3.39 (m, 1H), 3.20 (dd, J=12.5, 9.7 Hz, 1H).

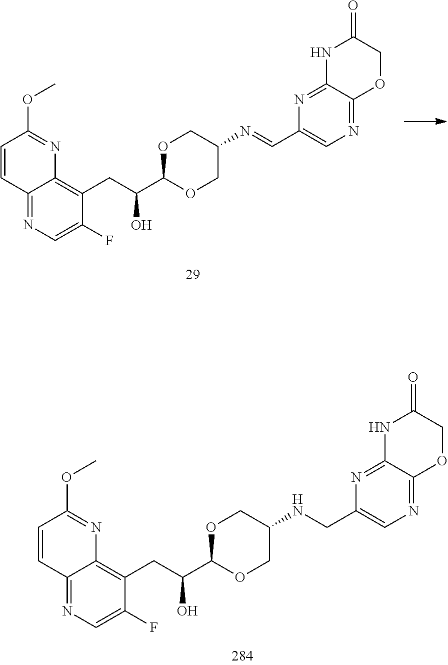 [SMILES: COc1ccc2ncc(F)c(C[C@H](O)[C@H]3OC[C@H](/N=C/c4cnc5c(n4)NC(=O)CO5)CO3)c2n1 ; COc1ccc2ncc(F)c(C[C@H](O)[C@H]3OC[C@H](NCc4cnc5c(n4)NC(=O)CO5)CO3)c2n1]

### 6-(((2-((S)-2-(3-fluoro-6-methoxy-1,5-naphthyridin-4-yl)-1-hydroxyethyl)-trans-1,3-dioxan-5-yl)amino)methyl)-2H-pyrazino[2,3-b][1,4]oxazin-3(4H)-one (284)

To a 250-mL RBF was added 6-((E)-((2-((S)-2-(3-fluoro-6-methoxy-1,5-naphthyridin-4-yl)-1-hydroxyethyl)-trans-1,3-dioxan-5-yl)imino)methyl)-2H-pyrazino[2,3-b][1,4]oxazin-3(4H)-one (29, 2.24 g, 4.63 mmol, 1 equiv) and a magnetic stir bar. Suspended in 46 mL of MeCN and set to stir at RT as sodium triacetoxyborohydride (1.96 g, 9.26 mmol, 2 equiv) was added in one portion at RT. To the heterogenous mixture was added TFA (1.58 g, 1.06 mL, 13.9 mmol, 3 equiv) across 1-2 minutes, mild exotherm observed. After stirring for 10 minutes, a small aliquot was removed and concentrated for evaluative NMR analysis. Evaluative NMR shows ~6:1 ratio of desired product to probably SM. After stirring for 20 minutes, another 0.5 mL of TFA was added. Reaction mixture took on a more vibrant yellow color and became more homogenous (still cloudy, but lots of specks went away). Poured into 92 mL of saturated bicarbonate solution and stirred at RT for ~5-10 minutes until gas evolution no longer apparent. Yellow golden, homogenous mixture was transferred to a 500-mL RBF and MeCN was removed (gently) on rotary evaporator. As MeCN was distilled off, a white precipitate began to form. Once no more MeCN distilled over, flask was removed and submerged in an ice-water bath. Added stir bar back and set to vigorously stir in the cold bath to fully precipitate desired product (appeared off white, but yellow supernatant could be masking this). After stirring for ~30 minutes, stored in fridge overnight. After standing overnight in fridge, collected precipitate via vacuum filtration on a Buchner funnel, rinsing flask and isolate with water. Allowed to dry on diaphragm pump, then dried in a lightly covered container in a vacuum desiccator with high vacuum overnight. Drying overnight in a vacuum desiccator afforded the title compound (1.862 g, 82.7%) as a soft, staticky, ivory powder. NMR of the isolated product shows desired product in high purity, baseline impurity present (~160:1 ratio of desired to impurity).

¹H NMR (DMSO-d₆, 400 MHz): δ 11.52 (br s, 1H), 8.73 (s, 1H), 8.26 (d, J=9.0 Hz, 1H), 7.73 (s, 1H), 7.21 (d, J=9.0 Hz, 1H), 4.82 (app s, 3H), 4.39 (d, J=3.9 Hz, 1H), 4.18-4.07 (m, 2H), 4.04-3.93 (m, overlaps with —OCH₃ peak, 1H), 4.02 (s, 3H), 3.71 (s, 2H), 3.43-3.21 (m, overlaps with water peak, 3H), 3.14 (dd, J=12.4, 9.9 Hz, 1H), 2.84-2.71 (m, 1H). 2.08 (br s, 1H).

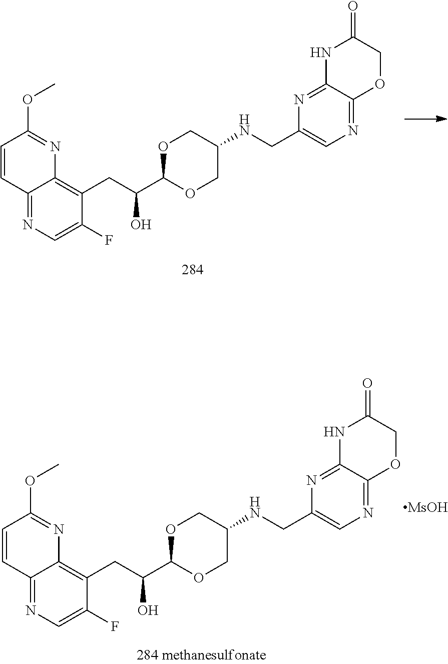

### 6-(((2-((S)-2-(3-fluoro-6-methoxy-1,5-naphthyridin-4-yl)-1-hydroxyethyl)-trans-1,3-dioxan-5-yl)amino)methyl)-2H-pyrazino[2,3-b][1,4]oxazin-3(4H)-one methanesulfonate salt (284 methanesulfonate)

To a 250-mL RBF was added 6-(((2-((S)-2-(3-fluoro-6-methoxy-1,5-naphthyridin-4-yl)-1-hydroxyethyl)-trans-1,3-dioxan-5-yl)amino)methyl)-2H-pyrazino[2,3-b][1,4]oxazin-3(4H)-one (284, 1.75 g. 3.6 mmol, 1 equiv) and a magnetic stir bar. Suspended in 36 mL of DCM, followed by 36 mL of MeOH. Upon addition of MeOH most of the solid had gone into solution, homogenous with ~1 minute of stirring to give a medium yellow solution. Cooled in an ice-water bath, then added a solution of MsOH in DCM (prepared from adding 0.236 mL of MsOH to 7.27 mL of DCM, 3.63 mmol, 1.01 equiv), portion-wise, across ~2-3 minutes. Allowed to stir for another 5-10 minutes in the ice bath, removed stir bar (washed with DCM) and concentrated reaction mixture gently on rotary evaporator to afford a tan foam that was manually scraped to afford a solid; however, some upper regions of flask still had a syrup or bits bound to the glass. Suspended in 21 mL of IPA and scraped the glass to remove all the product and dried on rotavap to afford a floral white solid. Scraped from sides of flask to afford a powder that was then returned to rotavap to dry under maximum vacuum (11-12 mbar) at 50° C. for 36 minutes, followed by drying under high vacuum. This afforded 2.009 grams of an eggshell-white solid. Dried overnight in a vacuum desiccator under high vacuum to afford the title compound (2.003 g) with trace amount of IPA. NMR shows desired product in a 23:1 molar ratio with IPA. Divided into 2 separate 1-gram batches and lyophilized from water (~1.2-1.4 mL per gram batch) over 48 hours. NMR shows minimal change in amount of remaining IPA.

¹H NMR (CD₃OD, 400 MHz): δ 8.63 (s, 1H), 8.21 (d, J=9.1 Hz, 1H), 7.88 (s, 1H), 7.17 (d, J=9.1 Hz, 1H), 4.98 (s, 2H), 4.62 (d, J=4.0 Hz, 1H), 4.53-4.43 (m, 2H), 4.30 (s, 2H). 4.30-4.22 (m, overlaps with other peak, 1H), 4.09 (s, 3H), 3.78 (t, J=10.7 Hz, 1H), 3.77 (t, J=10.8 Hz, 1H). 3.65-3.50 (m, 2H). 3.36 (dd, J=12.7, 9.0 Hz, overlaps with solvent peak, 1H), 2.70 (s, 3H).

UPLC-MS: 97.36%, 324 nm, found 487.1 m/z (M+H).

### Biological Activity

Compounds 147 (J. Med. Chem. 2021, 64, 15214) and 284 were prepared and tested. Results are shown in Table 1.

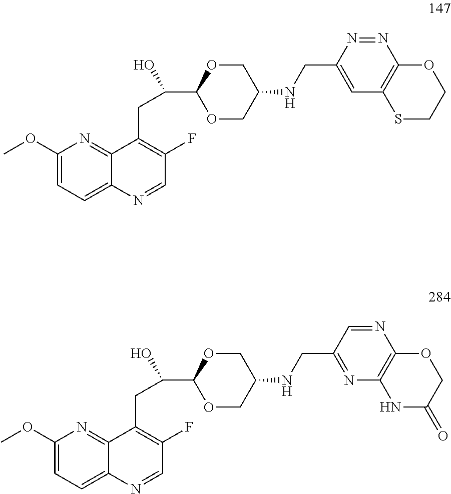 [SMILES: COc1ccc2ncc(F)c(C[C@H](O)[C@H]3OC[C@H](NCc4cc5c(nn4)OCCS5)CO3)c2n1 ; COc1ccc2ncc(F)c(C[C@H](O)[C@H]3OC[C@H](NCc4cnc5c(n4)NC(=O)CO5)CO3)c2n1]

TABLE 1

| Assay | 147 | | 284 | | 319 | |
|---|---|---|---|---|---|---|
| cLogP | 1.41 | | 0.57 | | 0.77 | |
| tPSA (Å²) | 119 | | 148 | | 114 | |
| FQ-R MRSA MIC₉₀ (μg/mL) | 0.25 | (n = 15) | 0.12 | (n = 25) | 0.5 | (n = 25) |
| D83N MIC (μg/mL) | 4-8 | | 1-2 | | 4 | |
| TopoIV/gyrase ratio | 16 | | 0.28 | | 0.65 | |
| hERG IC₅₀ (μM) | 103 | | 104 | | XX | |
| K562 IC₅₀ (μM) | 153 | | >200 | | 135 | |
| Mouse microsom. t_{1/2} (min) | 18-26 | | >>120 | | 347 | |
| Human microsom. t_{1/2} (min) | 94 | | >>120 | | >120 | |
| Rat microsom. t_{1/2} (min) | XX | | >>120 | | >120 | |
| Dog microsom. t_{1/2} (min) | XX | | >>120 | | >120 | |
| Monkey microsom. t_{1/2} (min) | XX | | >>120 | | >120 | |
| Mouse hepatocyte t_{1/2} (min) | XX | | >240 | | XX | |
| Human hepatocyte t_{1/2} (min) | XX | | >240 | | XX | |
| Mouse plasma t_{1/2} (min) | XX | | >120 | | >240 | |
| Human plasma t_{1/2} (min) | XX | | >120 | | 131 | |
| Rat plasma t_{1/2} (min) | XX | | >120 | | >240 | |
| Resistance freq. (at 8X MIC) | 2.02 × 10⁻9* | | <9.62 × 10⁻10** | | XX | |
| S. aur. MIC₉₀ (μg/mL) | 0.25 | (n = 30) | 0.25 | (n = 100) | 1 | (n = 100) |

| Mouse Cl_{unbound} (mL/min/kg) | 575 | 15*** | XX |
|---|---|---|---|
| | | 45**** | |
| Mouse PK Oral | 49 | 12-20*** | XX |
| bioavailability (%) | | 67**** | |

*Using S. aureus ATCC 29213.
**Using S. aureus ATCC 29213 and four additional strains of S. aureus.
***As free base in immunocompetent mice.
****As mesylate salt in neutropenic mice.

Additional data for Compounds 147 and 284, in comparison with gepotidacin, are shown in Table 2.

TABLE 2

| Assay | 147* | | 284* | | 319* | | gepotidacin* | |
|---|---|---|---|---|---|---|---|---|
| Staphylococcus aureus | 0.12/0.25 | (n = 30) | 0.12/0.25 | (n = 100) | 0.5/1 | (n = 100) | 0.25/0.5 | (n = 100) |
| MIC_{50/90} (μg/mL) | | | | | | | | |
| FQ-R MRSA MIC₉₀ (μg/mL) | 0.12/0.25 | (n = 15) | 0.12 | (n = 25) | 0.5/0.5 | | 0.25/0.5 | (n = 25) |
| Staphylococcus epidermidis | XX | | 0.12/0.25 | (n = 10) | XX | | 0.25/0.5 | (n = 10) |
| MIC_{50/90} (μg/mL) | | | | | | | | |
| Enterococcus spp. | 0.25/1 | (n = 10) | 1/2 | (n = 20) | 4/8 | (n = 20) | 2/4 | (n = 20) |
| MIC_{50/90} (μg/mL) | | | | | | | | |
| Streptococcus pneumoniae | 0.5/XX | (n = 5) | 0.5/1 | (n = 10) | 1/2 | (n = 10) | 0.12/0.25 | (n = 10) |
| MIC_{50/90} (μg/mL) | | | | | | | | |
| Streptococcus pyogenes | 0.5/XX | (n = 5) | 0.5/2 | (n = 10) | 2/8 | (n = 10) | 0.12/0.5 | (n = 10) |
| MIC_{50/90} (μg/mL) | | | | | | | | |
| Streptococcus agalactiae | 4/XX | (n = 5) | 4/16 | (n = 10) | 16/32 | (n = 10) | 0.5/1 | (n = 10) |

| | | | | | | |
|---|---|---|---|---|---|---|
| MIC_{50/90} (μg/mL) | | | | | | |
| Moraxella catarrhalis | XX | 0.12/0.25 | (n = 10) | XX | 0.06/0.06 | (n = 10) |
| MIC_{50/90} (μg/mL) | | | | | | |
| Haemophilus influenzae | XX | 4/8 | (n = 10) | XX | 1/2 | (n = 10) |

| MIC_{50/90} (μg/mL) | | | | | |
|---|---|---|---|---|---|
| Escherichia coli MIC₅₀ | XX | XX | 16 | (n = 8) | XX |
| (μg/mL) | | | | | |
| Pseudomonas aeruginosa | XX | XX | 16 | (n = 8) | XX |

| MIC₅₀ (μg/mL) | | | | | | | |
|---|---|---|---|---|---|---|---|
| Mycobacterium abscessus | XX | | >64/>64 | (n = 10) | XX | 16/64 | (n = 10) |
| MIC_{50/90} (μg/mL) | | | | | | | |
| Mycobacterium avium | XX | | 4 | (n = 4) | XX | 32 | (n = 4) |
| MIC₅₀ (μg/mL) | | | | | | | |
| Mycobacterium chimaera | XX | | 64 | (n = 6) | XX | >64 | (n = 6) |
| intracellulare group | | | | | | | |
| MIC₅₀ (μg/mL) | | | | | | | |
| Bacillus anthracis | 2/8 | (n = 17) | 0.06/0.25 | (n = 30) | XX | 0.5/0.5 | (n = 30) |

| MIC_{50/90} (μg/mL) | | | | | | |
|---|---|---|---|---|---|---|
| Francisella tularensis | XX | 0.5/4 | (n = 30) | XX | 0.12/0.5 | (n = 30) |
| MIC_{50/90} (μg/mL) | | | | | | |
| Yersinia pestis | XX | 2/8 | (n = 30) | XX | 0.12/0.5 | (n = 30) |
| MIC_{50/90} (μg/mL) | | | | | | |
| Burkholderia mallei | XX | 16/>64 | (n = 10) | XX | 8/≥64 | (n = 10) |
| MIC_{50/90} (μg/mL) | | | | | | |
| Burkholderia pseudomallei | XX | 32/64 | (n = 10) | XX | 32/32 | (n = 10) |

| MIC_{50/90} (μg/mL) | | | | | | |
|---|---|---|---|---|---|---|
| Neisseria gonorrhoeae | >4 | (n = 110) | 1/2 (n = 17) | XX | 0.5/1 | (n = 17) |

| MIC_{50/90} (μg/mL) | | 0.5/2 (n = 100) | | |
|---|---|---|---|---|
| Escherichia coli 25922 | >64 | 16 | 16 | 1-2 |
| MIC (μg/mL) | | | | |
| Acinetobacter baumannii | 64 | 8-16 | 16 | 32 |
| 19606 MIC (μg/mL) | | | | |
| Pseudomonas aeruginosa | >64 | 64 | 64 | 8-16 |
| 27853 MIC (μg/mL) | | | | |
| Mouse plasma protein binding | 12 | 31 | 27 | |
| (% unbound) | | | | |
| Human plasma protein binding | 15 | 10 | 23 | |
| (% unbound) | | | | |
| Rat plasma protein binding | XX | 4 | XX | |
| (% unbound) | | | | |
| Dog plasma protein binding | XX | 20 | XX | |
| (% unbound) | | | | |
| CYP inhibition at 10 μM (1A, | No issues | No issues | XX | |
| 2B6, 2C8, 2C9, 2C19, 2D6, 3A) | | | | |
| In vitro micronucleus | Not tested | Pos. at high | XX | |
| | | concentrations | | |
| Ames | Positive | Indeterminate | XX | |
| | (TA102) | | | |
| Kinetic solubility at pH | 69.6 | 117.5, >200 (as | 25 | |
| 7.4-7.5 (μM) | | mesylate salt) | | |
| Permeability: CACO-2 | 5.68 | 1.5 | 0.8 | |
| A−>B (×10⁻6 cm/s) | | | | |
| Permeability: MDCK II | XX | 0.74 | 1.96 | |
| A−>B (×10⁻6 cm/s) | | | | |
| Topological polar surface | 119 | 148 | 114 | |
| area (Å²) | | | | |
| Mouse PK Oral bioavailability | 49 | 12-20** | XX | |
| (%) | | 67*** | | |
| Mouse PK AUC_{last, unbound} | 0.9 | 5** | XX | |
| (μg*h/mL) after 50 mg/kg | | 12*** | | |
| oral dose | | | | |
| Mouse Vdss (L) | 1 | 0.36** | XX | |
| | | 0.53*** | | |
| Safety44 screen at 100 μM | 5-HT2b (72%) | None >50% | XX | |
| Comprehensive in vitro | No issues**** | No issues**** | No issues**** | |
| proarrhythmia assay at 100 μM | | | | |
| Cytotoxicity at 100 μM | No issues | No issues | No issues | |

*MIC_{50/90} values for 147 were obtained in separate studies. MIC_{50/90} values for 284 and gepotidacin were determined side-by-side in the same studies. MIC_{50/90} values for 319 were determined in a separate study from 284/gepotidacin but using the same strains for testing.
***As free base in immunocompetent mice.
****As mesylate salt in neutropenic mice.
****<25% inhibition at 100 μM test article concentration.

Compounds 284 and 319 were further tested. The results are shown in Table 3.

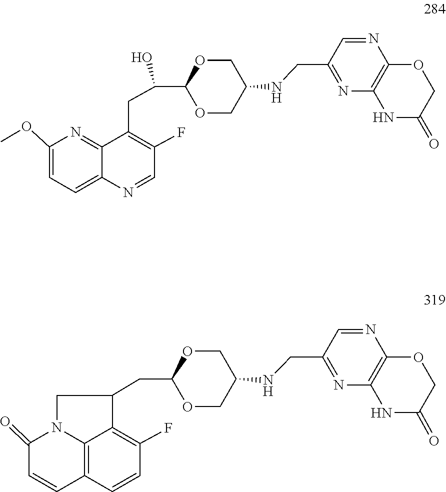 [SMILES: COc1ccc2ncc(F)c(C[C@H](O)[C@H]3OC[C@H](NCc4cnc5c(n4)NC(=O)CO5)CO3)c2n1 ; O=C1COc2ncc(CN[C@H]3CO[C@H](CC4Cn5c(=O)ccc6ccc(F)c4c65)OC3)nc2N1]

TABLE 3

| Compound | 284 | 319 |
|---|---|---|
| Molecular Weight | 486.46 | 467.46 |
| tPSA | 147.72 | 113.85 |
| CLogP | 0.57 | 0.77 |
| S. aureus 29213 MIC (μg/mL) | 0.125-0.25 | 0.5 |
| S. aureus USA300 MIC (μg/mL) | 0.125-0.25 | 0.5 |
| S. aureus CF isolate MIC (μg/mL) | 0.125-0.25 | 0.25 |
| D83N MIC (μg/mL) | 1-2 | 4 |
| M121K MIC (μg/mL) | 1-4 | 4 |
| TopoIV/gyrase ratio | 0.28 | 0.65 |
| Comprehensive in vitro | All <25% | All <25% |
| proarrhythmia assay at 100 μM | inhibition | inhibition |
| Mouse microsom. t_{1/2} (min) | >>120 | 347 |
| Human microsom. t_{1/2} (min) | >>120 | >120 |
| K562 IC₅₀ (μM) | >200 | 135 |
| MDCK II (284) A−>B (×10⁻6 cm/s) | 0.74 | 1.96 |

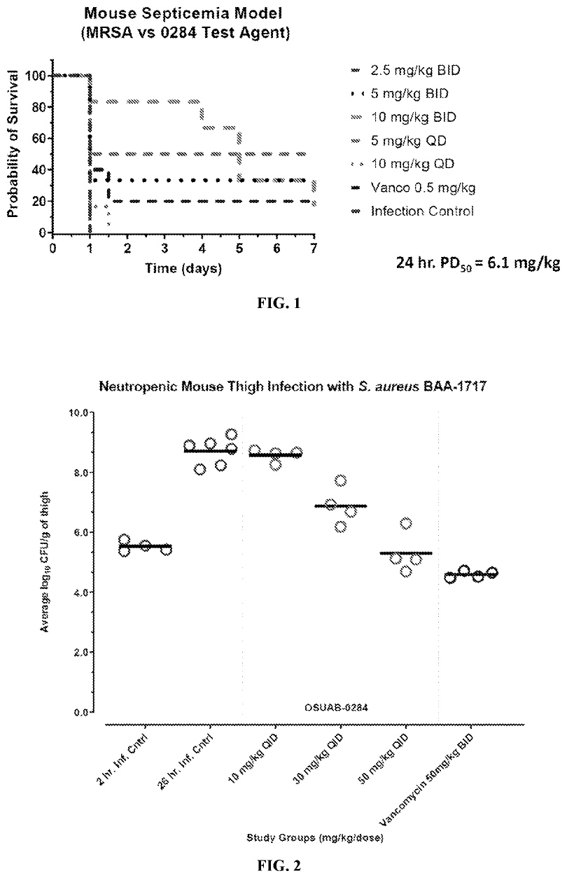

Additional data for Compounds 284 in a mouse septicemia model is shown in FIG. 1.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A Type II Topoisomerase inhibitor having Formula I: 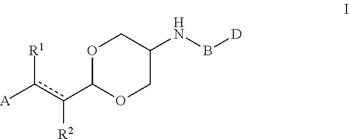
wherein
the dashed line represents a bond that is present or absent, and when the bond is present, R¹ and R² can be cis or trans;
A is a fused bicyclic aryl or bicyclic heteroaryl ring optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol or A and R¹ together form a tricyclic ring optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
B is C₁-C₆ alkyl or C₄-C₆ cycloalkyl optionally substituted with one or more oxo, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, phosphate, or thiol;
D is a heteroaryl ring having Formula IV 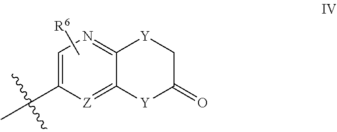
wherein
each Y is, independently, chosen from O, S, NH, or CH₂;
Z is N or CH; and
R⁶ is chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
R¹ and R² are, independently, chosen from H, OH, Cl, F, Br, I, CN, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol, or R¹ is a C₁-C₃ alkyl or C₂-C₃ alkenyl, optionally substituted with R¹⁰, also bound to A;
each R³ is, independently, chosen from C₁-C₆ alkyl, C₁-C₆ cycloalkyl, aryl, heteroaryl, heterocycloalkyl, and heteroalkyl, any of which are optionally substituted with C₁-C₆ alkyl, C₁-C₆ alkoxyl, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol; and
R¹⁰ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
or a pharmaceutically acceptable salt thereof.

2. The inhibitor of claim 1, wherein R¹ and R² are, independently, chosen from H, F, CN, OH, and NH₂.

3. The inhibitor of claim 1, wherein R² is NH₂, H, or OH.

4. (canceled)

5. The inhibitor of claim 1, wherein R¹ is H or OH.

6. The inhibitor of claim 1, wherein R¹ is OH and R² is OH.

7. The inhibitor of claim 1, wherein A is a fused bicyclic aryl or bicyclic heteroaryl ring having Formula II: 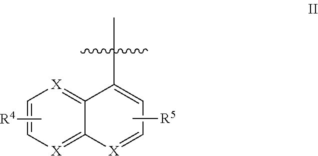
wherein
each X is, independently, CH or N; and
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

8. The inhibitor of claim 7, wherein R⁴ and R⁵ are, independently, chosen from H, Cl, F, Br, I, CN, OH, and unsubstituted C₁-C₆ alkyl or C₁-C₆ alkoxyl.

9. The inhibitor of claim 7, wherein R⁴ and R⁵ are, independently, chosen from H, Cl, F, OH, and methoxyl.

10. (canceled)

11. The inhibitor of claim 7, wherein two X's are N and the other X is CH.

12. The inhibitor of claim 7, wherein two X's are CH and the other X is N

13. The inhibitor of claim 1, wherein A is a fused bicyclic aryl or bicyclic heteroaryl ring having Formula III: 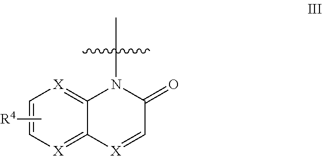
wherein
each X is, independently, CH or N;
R⁴ is chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

14. The inhibitor of claim 13, wherein R⁴ is chosen from H, Cl, F, Br, I, OH, and unsubstituted C₁-C₆ alkyl or C₁-C₆ alkoxyl.

15. The inhibitor of claim 13, wherein R⁴ is chosen from H, Cl, F, OH, and methoxyl.

16. (canceled)

17. The inhibitor of claim 1, wherein A and R¹ together have Formula IX, X, XI, or XII 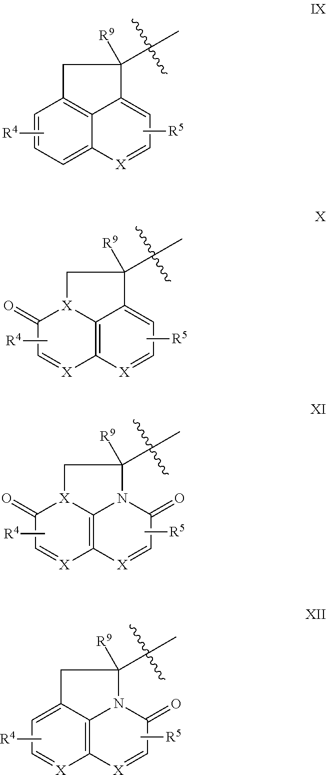
wherein
each X is, independently, CH, N, or CR¹;
R⁴ and R⁵ are, independently, chosen from H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, and C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol;
each R⁸ is Cl, F, CN, OH, OCH₃, CH₃, or NH₂; and
R⁹ is H, Cl, F, Br, I, CN, OH, NO₂, NH₂, CF₃, CO₂H, CO₂NH₂, CO₂NHR³, CO₂R³, C(O)R³, C(O)NH₂, C(O)NHR³, or C₁-C₆ alkyl or C₁-C₆ alkoxyl optionally substituted with alkoxy, alkenyl, alkynyl, aryl, heteroaryl, aldehyde, amino, carboxylic acid, ester, ether, halide, hydroxy, ketone, cyano, nitro, silyl, sulfo-oxo, sulfonyl, sulfone, sulfoxide, phosphate, or thiol.

18. The inhibitor of claim 17, wherein R⁴ is H and R⁵ is F.

19. The inhibitor of claim 1, wherein B is a C₁-C₆ alkyl, —C(═O)— or C₄-C₆ cycloalkyl chosen from unsubstituted methyl, ethyl, propyl, butyl, cyclobutyl, or cyclopentyl.

20. The inhibitor of claim 1, wherein B is CH₂ or cyclobutyl.

21. The inhibitor of claim 1, wherein R⁶ is chosen from H, Cl, F, Br, I, CN, OH, and unsubstituted C₁-C₆ alkyl or C₁-C₆ alkoxyl.

22. (canceled)

23. The inhibitor of claim 1, wherein one Y is S and the other is O.

24. The inhibitor of claim 1, wherein one Y is NH and the other is O.

25. (canceled)

26. (canceled)

27. (canceled)

28. (canceled)

29. The inhibitor of claim 1, wherein the compound is chosen from

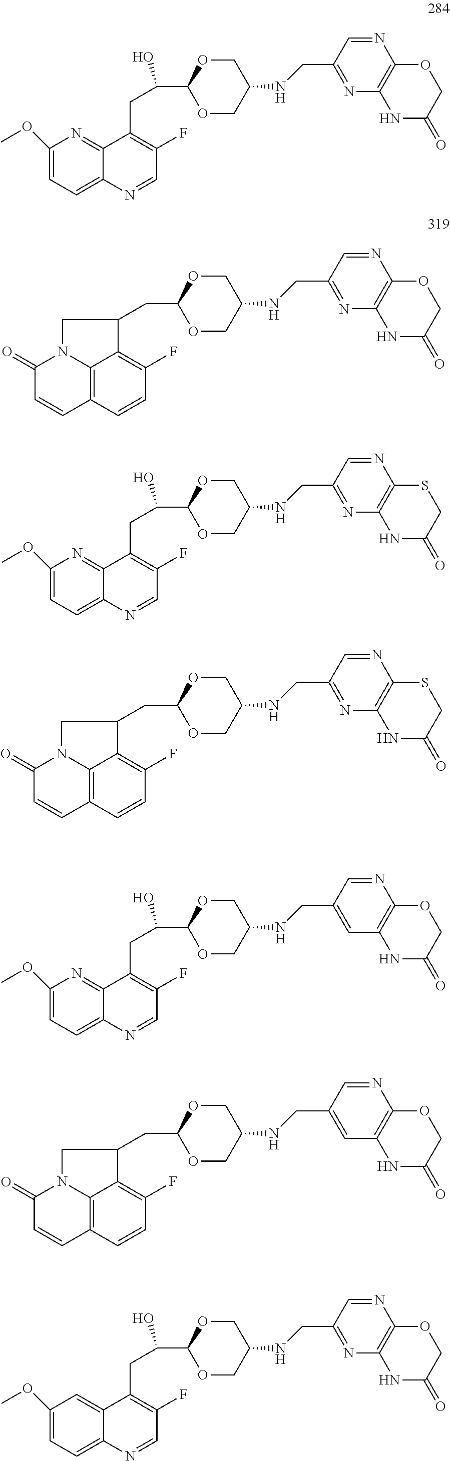

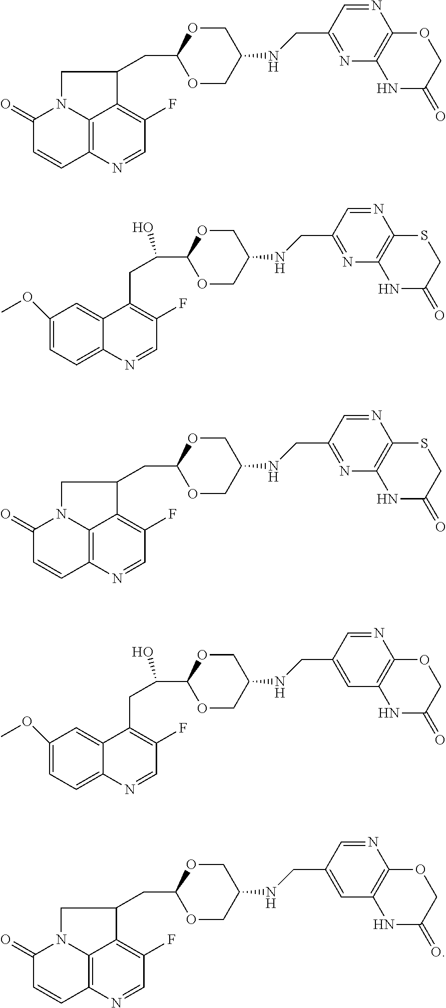 [SMILES: O=C1COc2ncc(CN[C@H]3CO[C@H](CC4Cn5c(=O)ccc6ncc(F)c4c65)OC3)nc2N1 ; COc1ccc2ncc(F)c(C[C@H](O)[C@H]3OC[C@H](NCc4cnc5c(n4)NC(=O)CS5)CO3)c2c1 ; O=C1CSc2ncc(CN[C@H]3CO[C@H](CC4Cn5c(=O)ccc6ncc(F)c4c65)OC3)nc2N1 ; COc1ccc2ncc(F)c(C[C@H](O)[C@H]3OC[C@H](NCc4cnc5c(c4)NC(=O)CO5)CO3)c2c1 ; O=C1COc2ncc(CN[C@H]3CO[C@H](CC4Cn5c(=O)ccc6ncc(F)c4c65)OC3)cc2N1]

30. A method of treating an infection in a subject caused by resistant Gram-positive bacteria, comprising: administering a therapeutically effective amount of the inhibitor of claim 1 to the subject.

31. The method of claim 30, wherein the Gram-positive bacteria is Methicillin Resistant S. aureus.

32. The method of claim 30, wherein the Gram-positive bacteria is M. tuberculosis, M. avium, or M. abscessus infection.
